(19) United States
(12) Patent Application Publication
(10) Pub. No.: US 2003/0175947 A1
(43) Pub. Date: Sep. 18, 2003

(54) Enhanced mixing in microfluidic devices

(76) Inventors: Robin Hui Liu, Chandler, AZ (US); Ralf Lenigk, Chandler, AZ (US); Pankaj Singhal, Pasadena, CA (US); Piotr Grodzinski, Chandler, AZ (US); Xunhu Dai, Gilbert, AZ (US); Roberta L. Druyor-Sanchez, Mesa, AZ (US)

Correspondence Address: DORSEY & WHITNEY LLP, Suite 3400, Four Embarcadero Center, San Francisco, CA, 94111-4187, US

(21) Appl. No.: 10/199,948

(22) Filed: Jul. 19, 2002

Related U.S. Application Data
(63) Continuation of application No. 09/993,342, filed on Nov. 5, 2001.

(51) Int. Cl. C12M 1/34; G01N 33/48

(52) U.S. Cl. 435/288.5; 436/174; 422/68.1

(57) ABSTRACT

The present invention provides microfluidic devices and methods for enhancing mixing and hybridization kinetics in microfluidic assays. More particularly, the present invention is a device and method wherein changing the volume of a gas pocket within a microfluidic device enhances mixing and reaction kinetics therein. In an embodiment sonic frequency is applied to the gas pocket resulting in microstreaming phenomena, thereby resulting in enhanced mixing and reaction kinetics. In another embodiment, the gas pocket is fluidly connected to a microfluidic channel and the volume of the pocket is changed (e.g., by heating and cooling of the gas therein), which cause oscillating flow within the microfluidic channel, thereby resulting in enhanced mixing and reaction kinetics therein.

### RELATED APPLICATIONS

This application claims the benefit under 35 U.S.C. §120 of copending application U.S. Ser. No. 091993,342, filed Nov. 5, 2001, hereby expressly incorporated by reference.

### FIELD OF THE INVENTION

The present invention is directed to mixing of fluids, including liquid solutions, within microfluidic devices. More specifically, the present invention provides methods and devices for enhancing mixing using acoustic, and particularly sonic, waves applied to a gas pocket within the microfluidic chamber. Additionally, the present invention provides using a gas pocket micropump to oscillate a fluid sample within a channel containing a probe array therein, thereby increasing sample exposure to the probes.

### BACKGROUND OF THE INVENTION

Rapid mixing is an essential process in many biochips and microfluidic systems used in bio-chemistry analysis, drug delivery, sequencing, synthesis of nucleic acids, and many others. Bisson, C., et al. A microanalytical device for the assessment of coagulation parameters in whole blood Solid-State Sensor and Actuator Workshop, Hilton Head S.C., 1998; Anderson, R. C., et al. Genetic Analysis Systems: Improvements and Methods Solid-State Sensor and Actuator Workshop, Hilton Head S.C., 1998; Chiem, N., et al. Microfluidic Systems for Clinical Diagnostics Transducers 97 (1997). Many biological processes such as DNA hybridization, cell activation, enzyme reactions, and protein folding demand rapid reactions that inevitably involve mixing of certain reactants. In DNA microarray hybridization mixing and binding of target DNA with immobilized probes is of fundamental importance. The hybridization reaction for most conventional DNA chips is diffusion-limited, and often takes a significant amount of time (˜18 hrs). It is widely believed and accepted that enhancing mixing, or otherwise enhancing sample/probe exposure time within the microfluidic reaction chamber can increase hybridization efficiency (i.e., reduce hybridization time), improve hybridization quality (i.e., uniformity), and reduce the amount of sample required.

Since turbulence is not practically attainable in micro-scale or mini systems with small dimensions (as these systems are limted to small Reynolds numbers, Re=(Q/A)Dh/v, where Q is the volumetric flow rate through the channel, A is the cross-sectional area, Dh is the hydraulic diameter of the channel, and v is the kinematic viscosity of the fluid), mixing in microfluidic systems is typically dominated by diffusion. Unfortunately, a pure diffusion-based mixing process can be very inefficient and often takes a long time, particularly when the sample solutions contain macromolecules (e.g., proteins and DNA) or large particles (e.g., bacteria or blood cells) that have diffusion coefficients orders of magnitude lower than that of most liquids. Therefore, an efficient micromixer is one way in which to enhance micromixing.

Commercial mixers are large (e.g., the Berger ball mixer used in BioLogic SFM4/Q Quenchflow machine is 1″×0.5″×0.5″). Berger, R. L., et al. High Resolution Mixer for the Study of Kinetics of Rapid Reactions in Solution Rev. of Scientific Instr. 39(4) (1968). Accordingly, they are unsuitable for use in most biochips and microfluidic systems. A few interesting micromixers have been developed in recent years. These include active micromixers that exert some form of active control over the flow field through such means as moving parts or varying pressure gradients (Evensen, H.T., et al. Automated Fluid Mixing in Glass Capillaries Rev. Scien. Inst. 69:519-526 (1998); Evans, J., et al. Plana Laminar Mixer MEMS (1997); Moroney, R. M., et al. Ultrasonically Induced Microtransport MEMS (1995)) and passive micromixers that utilize no energy input except the mechanism (pressure head or pump) used to drive the fluid flow at a constant rate. Branebjerg, J., et al. Fast Mixing By Lamination MEMS (1996); Mesinger, J., et al. Microreactor with Integrated Static Mixer and Analysis System MicroTAS (1994); Miyake, R., et al. Micro Mixer with Fast Diffusion MEMS (1993). One example of a passive micromixer is a multi-stage multi-layer laminarization scheme developed by Branebjerg et al. Branebjerg, J., et al. Fast Mixing By Lamination MEMS (1996). The mixer was designed to have two flow streams guided on top of each other and kept separated by a plate until they are forced to laminate (divide and mix). Enhanced mixing resulted from the increased contact area and decreased diffusion length when the two liquids were stacked. One of the limitations of such a device is the increased downstream pressure gradient associated with multiple divisions of the stream. Another disadvantage of using such a mixer in biomolecular analysis systems is the possibility of causing potential damage (e.g., denaturing) to biomolecules (e.g., large globular proteins). Such a micro-streaming scheme will force streams through smaller passages and orifices, and possibly create very high instantaneous rates-of-strain (e.g., shear and elongation) on biological macromolecules (e.g., globular proteins). High rate-of-strain fields can damage proteins and particles such as cells. Complex multi-subunit proteins are particularly prone to shear-induced loss of activity. Leckband, D. and Hammes, G. Interactions Between Nucleotide Binding Sites on Chloroplast Coupling Factor One During ATP Hydrolysis Biochem. 26:2306-2312 (1997). Moreover, increased surface-to-volume ratio of this mixer can lead to clogging and fouling caused by biomolecular adsorption onto the device surface.

Another passive micromixer example was developed by Miyake et al., using micro nozzles to inject one liquid into another making many micro-plumes. Miyake, R., et al. Micro Mixer with Fast Diffusion MEMS (1993). One limitation of such a mixer is energy loss. Additional energy is required for the injection. Other interesting passive mixing mechanisms used in some existing microfluidic systems include oscillation of a liquid plug in an i-STAT blood analyzer (Bisson, C., et al. A microanalytical device for the assessment of coagulation parameters in whole blood Solid-State Sensor and Actuator Workshop, Hilton Head S.C., 1998) and “meniscus recirculation mixing” in the GeneChip™ developed by Affymetrix Inc. (Anderson, R. C., et al. Genetic Analysis Systems: Improvements and Methods Solid-State Sensor and Actuator Workshop, Hilton Head S.C., 1998). In the former, a segment of blood is oscillated in the region of a flow channel coated with a reagent. The oscillation results in a fluid convection causing the reagent to mix into the blood sample within 8-17 seconds. The shear force generated by the oscillational flow is used to remove the reagent on the channel surface into solution. This technique is attractive since it is simple and does not require a specific micromixer. However, the oscillation convection does not generate a global mixing pattern, and might increase shearing on the blood cells near the channel wall. Meniscus recirculation mixing (developed by Anderson et al.) generally works better in micro-scale if two liquids with different viscosity are mixing.

Examples of active micromixers include those of Moroney et al. Ultrasonically Induced Microtransport MEMS (1995) and Zhu et al. Microfluidic Motion Generation with Acoustic Waves Sensors and Actuators: A. Physical, v. 66 pp. 355-360 (April 1998). The former used ultrasonic traveling waves generated by a pieozoelectric film to the liquid in a mixing chamber. The latter used loosely-focused acoustic waves generated by an electrode-patterned pieozoelectric film. Both devices require a thin chamber wall (˜10 μm) between the liquid solution and the pieozoelectric film, resulting in a complicated and time-consuming fabrication process (e.g., Si bulk etching). The devices use ultrasonic frequency, generally above 20 kHz, which may disaggregate bacteria (William, A. R. and Slade, J. S. Ultrasonic Dispersal of Aggregates of Sarcina lutea Ultrasonics 9:85-87 (1971)) disrupt human erythrocytes and platelets in vitro and in vivo (William, A. R., et al. Hemolysis Near a Transversely Oscillating Wire Science 169:871-873 (1974); William, A. R., Intravascular Mural Thrombi Produced by Acoustic Microstreaming Ultrasound Med. Biol. 3:191-203 (1977)), or cause other bioeffects (Rooney, J. A., Shear as a mechanism for Sonically-Induced Biological Effects J. Acoust. Soc Am, 52:1718-1724 (1972); Clarke P. R. and Hill C. R., Physical and Chemical Aspects of Ultrasonic Disruption of Cells J. Acoust. Soc. Am. 50:649-653 (1970)). Zhu's device must use an open chamber, which limits its use in many applications that require enclosed chambers.

Another example of active micromixers is the bubble mixer demonstrated by Evans et al. Evans, J., et al. Plana Laminar Mixer MEMS (1997). The mixer uses bubble pumps by boiling a liquid at micromachined polysilicon and aluminum trace heaters to agitate the bulk liquid and create chaotic advection within the same. The complexity of this bubble heating scheme makes the device difficult to build and operate.

Three additional mixing methods were disclosed by Affymetrix Inc. for facilitating the mixing of various fluids within a DNA hybridization chamber. One is called “rotational mixing” that involves a rotatable body. U.S. Pat. No. 6,050,719. When rotating the chamber about the rotational axis, the fluid within the chamber allegedly becomes agitated on the theory that the direction of flow is hindered due to the change in direction of the chamber walls. However, when the reaction chamber is shallow (e.g., ˜200 μm deep), the inertial force of the fluid within the chamber is negligible due to low Reynolds number. In such a case, viscous forces dominate fluidic behavior (water behaves like honey in a shallow chamber). It is believed that mixing in this case can not be significantly enhanced by mechanical agitation means, such as rotation or lateral shaking of the chamber, without providing some mechanism for the fluid within the chamber to move more freely within the chamber (such as and without limitation a flexible membrane, as disclosed in U.S. Provisional Appln. No. 60/308,169, filed Jul. 26, 2001). In addition, many difficulties arise in producing a functional device incorporating such agitation. For example, fluid connections should be provided from a flexible material, allowing movement of the chamber without translation of that motion to elements external to the chamber. As such, incorporating temperature control and monitoring into the hybridization arrays is challenging.

In the second of the three additional devices disclosed by Affymetrix Inc., Anderson et al., in U.S. Pat. No. 6,168,948, disclosed an acoustic mixing device that uses a PZT element (element composed of lead, zirconium and titanium containing ceramic) or lithium niobate in contact with the exterior surface of the hybridization chamber. Application of a current to this element generates ultrasonic vibrations that are translated to the reaction chamber resulting in mixing of the sample disposed therein. The vibrations of this element result in convection flow being generated within the reaction chamber. Since the PZT crystal was driven at 2 MHz, this acoustic mixing is very similar to Moroney's (Moroney, R. M., et al. Ultrasonically Induced Microtransport MEMS (1995)) and Zhu's (Zhu et al. Microfluidic Motion Generation with Acoustic Waves Sensors and Actuators: A. Physical, v. 66 pp. 355-360 (April 1998)) active mixers discussed above, and therefore the same problems exist. Moreover, an average power of 3 W was used by the device of Anderson et al., which may potentially heat up the hybridization solution.

In the third of the three additional devices disclosed by Affymetrix Inc., Besemer et al. in U.S. Pat. No. 6,114,122 disclosed two mixing approaches: bubbling gas through the chamber and “drain and fill” method. The former approach involves the flowing of an inert gas stream through the inlet port and out through the outlet port of the chamber. The latter method involves alternately reversing the direction of the system's pump to drain and then fill the chamber. Both mixing schemes suffer from the drawback that complicated system setups with in-line flowing and pumping are required. A precise flow control is necessary, which makes the operation complicated.

Therefore, there remains a need in the art for a microfluidic device in which mixing within the device is significantly enhanced, and which is relatively easy to manufacture and is relatively uncomplicated to operate.

### SUMMARY OF THE INVENTION

The present invention provides methods and microfluidic devices for mixing a sample within microfluidic cavity and for detecting a target analyte within the sample. An embodiment of the present invention is a method of mixing a sample in a microfluidic cavity comprising introducing the sample into a device comprising a microfluidic cavity. The microfluidic cavity has at least one gas pocket, and a substrate comprising at least one biological binding molecule. After the sample has been introduced to the microfluidic cavity, the volume of said gas pocket is altered to mix said sample such that said target analyte binds to said biological binding molecule. In an alternative embodiment, the gas pocket further comprises a heater and said altering is done by heating and cooling said gas pocket. In another embodiment the altering is done by the application of sonic waves to said microfluidic cavity, generally, but not always, having a frequency less than 20 kHz. In an alternative embodiment the device further comprises a PZT device (preferably a PZT film) and the altering is done by oscillating the gas pocket with ultrasonic waves. It will be appreciated that the cavity may contain a plurality of biological binding molecules therein and/or comprise a channel with biological binding molecules (sometimes referred to herein as “capture binding ligands” or “capture probes”). In some embodiments, the biological binding molecules may be attached to a plurality of electrodes to facilitate electronic detection of the target analytes.

### DESCRIPTION OF THE DRAWINGS

FIG. 1 depicts a view of a microfluidic device according to an embodiment of the present invention;

FIG. 2 depicts a cross-section view of a microfluidic device according to an embodiment of the present invention;

FIG. 3 depicts a microfluidic device according to another embodiment of the present invention;

FIG. 4 is an image of an embodiment of the present invention;

FIGS. 5-7 are images of the embodiment of FIG. 4 at different stages of an experiment;

FIG. 8 depicts an illustration of one possible mechanism for enhanced mixing within an embodiment of the present invention, although it is to be understood that this illustration is not presented by way of limitation of how the invention may work;

FIGS. 9-12 are images of an alternative embodiment of the present invention at different stages of an experiment;

FIGS. 13-16 are images of an alternative embodiment of the present invention at different stages of an experiment;

FIGS. 17-20 are images of an alternative embodiment of the present invention at different stages of an experiment;

FIGS. 21 and 22 are fluorescent images of a 2-oligo hybridization assay in a 4-up biochip, with the assay of FIG. 21 being performed in diffusion based chip and FIG. 22 being performed in an embodiment of the present invention;

FIG. 23 presents data comparing averaged fluorescent intensity data of the assays of FIGS. 21 and 22;

FIG.24 presents data comparing the intensity uniformity for the two assays of FIGS. 21 and 22;

FIGS. 25-28 are images of an alternative embodiment of the present invention at different stages of an experiment;

FIG. 29 shows gel electrophoresis data from an assay conducted in an embodiment of the present invention: lane 1 is an HFE-H amplicon; lane 2 is a negative control; and lane 3 is 100 bp DNA ladder;

FIG. 30 summarizes the hybridization kinetics results for an assay on an array in two different alternative embodiments of the present invention and that in a diffusion-based array; and FIG. 31 summarizes data for an HFE-H assay in an embodiment of the present invention and that in a diffusion-based array where 70 bp DNA oligos served as target mimic in place of the amplicon used in the experiment summarized in FIGS. 29 and 30.

### DETAILED DESCRIPTION OF THE PREFERRED EMBODIMENTS

The present invention discloses a novel device and method for enhancing mixing within a microfluidic cavity. In an embodiment of the present invention, the device and method are based on the principle of bubble-induced acoustic microstreaming. More particularly, an embodiment of the present invention is directed to a microfluidic system having a microfluidic cavity, wherein at least one gas pocket is present in a liquid solution within the microfluidic cavity. The gas pocket may be formed by introducing a gas bubble directly into the liquid solution or providing an indentation within the microfluidic cavity wall such that a gas pocket will form between the liquid and the wall upon the introduction of the liquid solution into the microfluidic cavity. The gas pocket is then expanded and contracted by application of sonic frequency to and around the gas pocket, ultimately resulting in convection flows, and, thus, rapid mixing within the microfluidic chamber.

In an alternative embodiment, the gas pocket is fluidly connected to the microfluidic chamber, preferably a microfluidic channel. Expansion and contraction of the gas pocket or gas therein (for example and not by way of limitation, by heating and cooling of the gas therein) causes the fluid to oscillate back and forth within the microfluidic channel, thereby resulting in increased contact between molecules within the liquid, and, for example, biological binding molecules attached to the surface of microfluidic channel.

Accordingly, the present invention provides methods and microfluidic devices for mixing a sample within microfluidic cavity and, optionally, for detecting a target analyte within the sample.

In some embodiment, methods and devices provided by the present invention are directed toward detecting target analytes in a sample. As will be appreciated by those in the art, the sample solution may comprise any number of things, including, but not limited to, bodily fluids (including, but not limited to, blood, urine, serum, lymph, saliva, anal and vaginal secretions, perspiration and semen; and solid tissues, including liver, spleen, bone marrow, lung, muscle, brain, etc.) of virtually any organism, with mammalian samples being preferred and human samples being particularly preferred); environmental samples (including, but not limited to, air, agricultural, water and soil samples); biological warfare agent samples; research samples (i.e. in the case of nucleic acids, the sample may be the products of an amplification reaction, including both target and signal amplification as is generally described in PCT/US99/01705, such as PCR or SDA amplification reactions); purified samples, such as purified genomic DNA, RNA, proteins, etc.); raw samples (bacteria, virus, genomic DNA, etc.; As will be appreciated by those in the art, virtually any experimental manipulation may have been done on the sample.

Suitable target analytes include organic and inorganic molecules, including biomolecules. In a preferred embodiment, the analyte may be an environmental pollutant (including pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eukaryotic cells, including mammalian tumor cells); viruses (including retroviruses, herpesviruses, adenoviruses, lentiviruses, etc.); and spores; etc. Particularly preferred analytes are environmental pollutants; nucleic acids; proteins (including enzymes, antibodies, antigens, growth factors, cytokines, etc); therapeutic and abused drugs; cells; and viruses.

In a preferred embodiment, the analyte is a nucleic acid. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506,and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. Nucleic acid analogs also include “locked nucleic acids”. All of these references are hereby expressly incorporated by reference. These modifications of the ribose-phosphate backbone may be done to facilitate the addition of labels, etc., or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

As outlined herein, the nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as nucleosides.

In a preferred embodiment, the present invention provides methods of manipulating and detecting target nucleic acids. By “target nucleic acid” or “target sequence” or grammatical equivalents herein means a nucleic acid sequence, generally on a single strand of nucleic acid. The target sequence may be a portion of a gene, a regulatory sequence, genomic DNA, cDNA, RNA including mRNA and rRNA, or others. It may be any length, with the understanding that longer sequences are more specific. In some embodiments, it may be desirable to fragment or cleave the sample nucleic acid into fragments of 100 to 10,000 basepairs, with fragments of roughly 500 basepairs being preferred in some embodiments. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others.

As is outlined more fully below, probes (including primers) are made to hybridize to target sequences to determine the presence or absence of the target sequence in a sample. Generally speaking, this term will be understood by those skilled in the art.

The target sequence may also be comprised of different target domains, which may be adjacent (i.e. contiguous) or separated. For example, when oligonucleotide ligation amplification (OLA) reaction techniques are used, a first probe or primer may hybridize to a first target domain and a second primer may hybridize to a second target domain; either the domains are adjacent, or they may be separated by one or more nucleotides. The terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain.

In a preferred embodiment, the analyte is a protein. As will be appreciated by those in the art, there are a large number of possible proteinaceous target analytes that may be detected using the present invention. By “proteins” or grammatical equivalents herein is meant proteins, oligopeptides and peptides, derivatives and analogs, including proteins containing non-naturally occurring amino acids and amino acid analogs, and peptidomimetic structures. The side chains may be in either the (R) or the (S) configuration. In a preferred embodiment, the amino acids are in the (S) or L-configuration. As discussed below, when the protein is used as a binding ligand, it may be desirable to utilize protein analogs to retard degradation by sample contaminants.

Suitable protein analytes include, but are not limited to, (1) immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, a-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators ( theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C.perfringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lambliay Y. pestis, Pseudomonas, e.g. P. aeruginosa, P. putida; Chlamydia, e.g. C. trachomatis; Bordetella, e.g. B. pertussis; Treponema, e.g. T. palladium; and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphotase; aldolase, prostatic acid phosphatase, terminal deoxynucleotidyl transferase, and bacterial and viral enzymes such as HIV protease; (3) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone and testosterone; and (4) other proteins (including a-fetoprotein, carcinoembryonic antigen CEA, cancer markers, etc.).

In addition, any of the biomolecules for which antibodies may be detected may be detected directly as well; that is, detection of virus or bacterial cells, therapeutic and abused drugs, etc., may be done directly.

Suitable analytes include carbohydrates, including but not limited to, markers for breast cancer (CA15-3, CA 549, CA 27.29), mucin-like carcinoma associated antigen (MCA), ovarian cancer (CA125), pancreatic cancer (DE-PAN-2), prostate cancer (PSA), CEA, and colorectal and pancreatic cancer (CA 19, CA 50, CA242).

The present invention provides microfluidic devices with mixing components. By ‘microfluidic devices’ herein is meant a device suitable for handling small amounts of fluid, generally nanoliters, although in some applications a larger or smaller fluid volume will be necessary. Structures within such microfluidic devices generally have dimensions on the order of microns, although in many cases larger dimensions on the order of millimeters, or smaller dimensions on the order of nanometers, are advantageous.

As will be appreciated by those in the art, the microfluidic devices of the present invention may be fabricated in a variety of ways and may be substantially composed of a variety of materials. A variety of suitable materials, methods and configurations are described in WO 00/62931, WO 01/54813 and PCT US99/23324, all of which are expressly incorporated by reference herein in their entirety.

As is known in the art, microfluidic devices are generally constructed substantially of a substrate. The substrate can be made of a wide variety of materials and can be configured in a large number of ways, as is discussed herein and will be apparent to one of skill in the art. The composition of the substrate will depend on a variety of factors, including the techniques used to create the device, the use of the device, the composition of the sample, the analyte to be detected, the size of internal structures, the presence or absence of electronic components, etc. Generally, the devices of the invention should be easily sterilizable as well, although in some applications this is not required.

In a preferred embodiment, the substrate can be made from a wide variety of materials including, but not limited to, silicon such as silicon wafers, silicon dioxide, silicon nitride, glass and fused silica, gallium arsenide, indium phosphide, aluminum, ceramics, polyimide, quartz, plastics, resins and polymers including polymethylmethacrylate, acrylics, polyethylene, polyethylene terepthalate, polycarbonate, polystyrene and other styrene copolymers, polypropylene, polytetrafluoroethylene, superalloys, zircaloy, steel, gold, silver, copper, tungsten, molybdeumn, tantalum, KOVAR, KEVLAR, KAPTON, MYLAR, brass, sapphire, etc. High quality glasses such as high melting borosilicate or fused silicas may be preferred for their UV transmission properties when any of the sample manipulation steps require light based technologies. In addition, as outlined herein, portions of the internal surfaces of the device may be coated with a variety of coatings as needed, to reduce nonspecific binding, to allow the attachment of binding ligands, for biocompatibility, for flow resistance, etc.

Microfluidic devices of the present invention may be fabricated using a variety of techniques, including, but not limited to, hot embossing, such as described in H. Becker, et al., Sensors and Materials, 11, 297, (1999), hereby incorporated by reference, molding of elastomers, such as described in D. C. Duffy, et. al., Anal. Chem., 70, 4974, (1998), hereby incorporated by reference, injection molding, silicon fabrication and related thin film processing techniques, circuit board fabrication technology, and in a preferred embodiment, the microfluidic devices are fabricated using ceramic multilayer fabrication techniques, such as are outlined in PCT/US99/23324 U.S.Ser. Nos. 09/235,081; 09/337,086; 09/464,490; 09/492,013; 09/466,325; 09/460,281; 09/460,283; 09/387,691; 09/438,600; 09/506,178; and 09/458,534; all of which are expressly incorporated by reference in their entirety. In this embodiment, the devices are made from layers of green-sheet that have been laminated and sintered together to form a substantially monolithic structure.

Microfluidic devices of the present invention may contain a variety of structures for containing fluid, either stationary fluid or flowing fluid. These structures fall generally into two categories, referred to herein as chambers and channels. By ‘chamber’, herein is meant a space or volume that is capable of containing a volume of fluid. In some embodiments, chambers are provided for the storage of agents or samples. In some embodiments, chambers are provided allowing sample fluid to contact an electrode, a physical constriction, or a detection module, as described further below. A chamber can be any shape, for example it may be square, rectangular, cylindrical, or the like. It may connect with other chambers. Chambers may be closed and completely internal to the device, or may be open to some degree to allow the introduction of sample. The volume of a chamber can vary depending on the fluid it is designed to contain and the application. In general, chamber sizes range from 1 nL to about 1 mL, with from about 1 to about 250 μL being preferred and from about 10 to about 100 μL being especially preferred. By ‘channel’, or ‘microchannel’, herein is meant a space capable of containing a volume of fluid within the device. Generally, ‘channel’ or ‘microchannel’ refers to a region designed to have fluid moved through it, substantially from one end of the channel to another. In some embodiments, channels are designed to allow fluid to come into contact with an electrode, a physical constriction or a detection module, as described further below. A channel may have any shape, for example, it may be linear, serpentine, arc shaped and the like. The cross-sectional dimension of the channel may be square, rectangular, semicircular, circular, etc. Additionally, the cross-sectional dimension of the channel may change across its length. Channels may be closed and completely internal to the device, or they may be substantially open to accommodate the introduction or removal of sample or agents. The channels have preferred depths on the order of 0.1 μm to 100 μm, typically 2-50 μm. The channels have preferred widths on the order of 2.0 to 500 μm, more preferably 3-100 μm. For many applications, channels of 5-50 μm are useful. In one embodiment, a channel with a 200 μm cross-section is provided. There may be multiple and interconnected channels. In one embodiment of the present invention, channels in one orientation intersect at multiple locations with channels having an orthogonal orientation.

Microfluidic devices comprising chambers and channels may be fabricated in a variety of ways depending on the size, orientation and intended use of the channels and chambers as well as their material composition.

In a preferred embodiment, chambers, channels, or the substrate of the microfluidic device are made from, or coated with, biocompatible materials in regions where they will come into contact with biological samples. In particular, materials that provide a surface that retards the non-specific binding of biomolecules, e.g. a “non sticky” surface, are preferred. For example, when a chamber is used for PCR or amplification reactions a “non sticky” surface prevents enzymatic components of the reaction mixture from sticking to the surface and being unavailable in the reaction.

Biocompatible materials are well known in the art and include, but are not limited to, plastic (including acrylics, polystyrene and copolymers of styrene and other materials, polypropylene, polyethylene, polybutylene, polyimide, polycarbonate, polyurethanes, Teflon™, and derivatives thereof, etc.) Other configurations include combinations of plastic and printed circuit board (PCB; defined below). For example at least one side of a chamber is printed circuit board, while one or more sides of a chamber are made from plastic. In a preferred embodiment, three sides of a chamber are made from plastic and one side is made from printed circuit board. In addition, the chambers, channels, and other components of the systems described herein may be coated with a variety of materials to reduce nonspecific binding. These include proteins such as caseins and albumins (bovine serum albumin, human serum albumin, etc.), parylene, other polymers, etc.

In general, the microfluidic device includes at least one microfluidic cavity. As used herein “microfluidic cavity” means microfluidic chamber, microfluidic channel or any void within a microfluidic device in which mixing or target analyte detection is desired. More specifically, it is preferred that the a microfluidic cavity have a volume of 100 nanoliters to 250 microliters.

In a preferred embodiment, the microfluidic devices of the invention comprise a mixing module comprising mixing components as outlined herein. In a preferred embodiment, the mixing module is used both as a mixing module and as a detection module, such that the mixing occurs within the detection module and decreases the assay time of detection. Thus, while the majority of the discussion herein is directed to mixing modules that additionally serve as detection modules, those in the art will appreciate that microfluidic devices comprising mixing modules that do not involve detection are also included.

Accordingly, in a preferred embodiment, the microfluidic devices of the invention comprise a detection module that comprises an array of capture binding ligands, described below, and mixing components or mixing capability. As outlined herein, there are several preferred mixing components generally relying on the incorporation of at least one air bubble or pocket that is used for mixing.

In a preferred embodiment, the mixing components include at least one air bubble into the detection module, which is then acoustically manipulated to vibrate, which allows better mixing and motion of the fluid within the chamber, thus allowing better contact between the capture binding ligands on the array and the target analytes in solution. This in turn allows faster binding kinetics and reduced assay times.

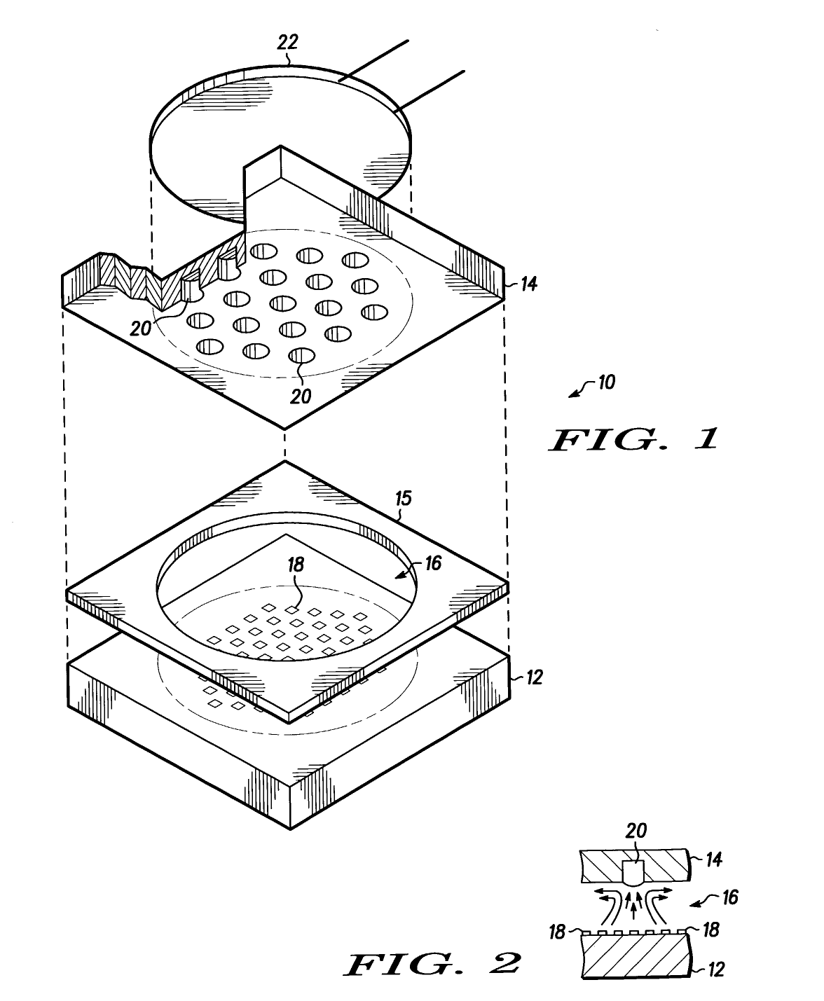

This system is generally depicted in FIG. 1. Referring to FIG. 1 microfluidic system 10, in accordance with an embodiment of the present invention, has substrate 12, top layer 14 and adhesive layer 15, which define microfluidic chamber 16. Preferably, substrate 12 has one or more microarrays 18, which are more thoroughly discussed above. In a preferred embodiment, indentations 20 are inserted into top layer 14, thereby providing indentations within microfluidic chamber 16, which trap gas (preferably air) upon introduction of a liquid sample into microfluidic chamber 16. In this manner a gas pocket is formed between the indentation and the liquid sample within the microfluidic chamber. Preferably, indentations 20 are micromachined into the walls of microfluidic chamber 16, e.g., in top layer 14, substrate 12 or both. A skilled artisan will recognize many different ways to form a gas pocket, all of which fall within the scope of the present invention. A schematic cross-sectional view of microfluidic chamber 16 within system 10 is shown in FIG. 2. Chamber 16 is bordered by substrate 12 and top layer 14 containing indentation 20. Microarrays 18 are formed on substrate 12.

Referring back to FIG. 1, preferably, a PZT device, for example a PZT disk 22 is attached to a surface of the device. A preferred embodiment utilizes attachment to the top layer 14; however, as will be appreciated by those in the art, attachment to other surfaces, like the bottom surface, substrate, the walls, etc., is also possible. The PZT disk is used to apply sonic waves to microfluidic chamber 16, including the gas pockets within microfluidic chamber 16. It will be appreciated that application of the sonic waves to and around the gas pockets (as distinguished from the entire cavity) will be sufficient and will fall within the scope of the present invention, although either option may be used. It will be further appreciated that any number of mechanisms used to apply sonic waves to the microfluidic chamber will fall within the scope of the present invention. For example and without limitation sonic waves may be applied using a sonic bath or sonic horn. Additionally, a PZT membrane may be used, and the membrane may be laminated with a polymer material.

Under a sonic frequency (e.g., approximately 4 kHz), flow circulation occurs around the gas pockets, resulting in convection flow, and, thus, rapid mixing. As a result, the mixing time to fully mix a 50 μL chamber is significantly reduced from hours (pure diffusion-based mixing) to approximately tens of seconds. Preliminary array tests, described more fully below, demonstrated that this bubble-induced acoustic mixing greatly improves hybridization sensitivity and uniformity compared to pure diffusion-based (static) hybridization within the same reaction time. Due to the low frequency (˜kHz) and low voltage (5 V peak-to-peak), the power consumption (˜2 mW) of the mixing apparatus is much lower than the prior art acoustic-wave mixers. The frequency used is generally dependent on the resonant frequency of the gas pocket. In some preferred embodiments of the invention, an air bubble having a diameter of 0.5-1 mm is used, resulting in the use of frequencies between 1-6 kHz. Th frequency may also be swept through a frequency range. An additional advantage over the prior art is that the transducer adds almost no heating to the liquid, is mechanically simple, and relatively easy/inexpensive to manufacture. This method and apparatus are particularly attractive for handheld electronic driven DNA analysis instrument.

Without wishing to be bound by any particular theory, a gas pocket in a liquid medium acts as an actuator (i.e., the surface of the gas pocket behaves like a vibrating membrane) when the pocket undergoes expansion and contraction within a sound field. The behavior of a gas pocket in sound fields is determined largely by its resonance characteristic. For frequencies in the range considered in the present disclosure (˜kHz) the radius of a pocket resonant at frequency f (Hz) is given by the equation:

2πf=\sqrt{3γP₀lρ} (1)

where a is the pocket radius (cm), γ is the ratio of specific heats for the gas and liquid, P₀, is the hydrostatic pressure (dynes/cm²), and ρ is the density of the liquid (g/cm³). Using this equation, and assuming an air/water interface with the parameter values f=5000 Hz, γ=1.4, P₀=106 dynes/cm² and ρ=1.0 g/cm³ one finds the radius a for resonance to be 0.65 mm.

Again without wishing to be bound by any particular theory, it is believed that when the pocket undergoes volume change within a sound field, the frictional forces between the boundary and the liquid medium result in a bulk fluid flow around the pocket, called cavitation microstreaming or acoustic microstreaming. Nyborg, W. L., Acoustic Streaming Near a Boundary J. Acoust. Soc. Am. 30:329-339 (1958). It is also believed, supported by experimental evidence provided below, that cavitation microstreaming within a microfluidic chamber is orderly at low driving amplitudes when the insonation frequency drives the pockets at their resonance frequency for pulsation, and when the pockets are situated on solid boundaries, e.g., against a wall of the microfluidic chamber.

The pocket-induced streaming is strongly dependent on frequency for a given bubble size, and on bubble radius for a given frequency. The motion is most rapid when the radius and frequency are related approximately by Eq. (1). A variation in either frequency (for fixed radius) or radius (for fixed frequency) from the conditions for maximum motion causes the streaming to be inappreciable. Acoustic microstreaming, arising about a single pocket excited close to resonance, produces strong liquid circulation flow in the microfluidic chamber. Streaming takes place at the liquid-gas interphase boundary causing a tangential liquid motion along the boundary. This liquid circulation flow can be used to effectively enhance mixing. The preferred sizes of the gas pocket are between 0.5 and 1 mm in diameter, and between 0.5 and 1 mm in height.

Without wishing to be bound by any particular theory, it is believed that the most effective mixing enhancement is provided by particular excitation frequencies generated by a desired number of gas pockets having a size selected in accordance with the resonant frequency of the acoustic transducer, preferably a PZT transducer. These theories are presented for the purposes of a possible explanation for how the present invention achieves enhanced mixing results, and are not to be used to or understood to limit the scope of the present invention.

In addition to microstreaming, as discussed above, in an alternative embodiment of the present invention, the gas pocket is fluidly connected to the microfluidic chamber, preferably a microfluidic channel. Expansion and contraction of the gas pocket or gas therein (for example and not by way of limitation, by heating and cooling of the gas therein) causes the fluid to oscillate back and forth within the microfluidic channel, thereby resulting in increased contact between molecules within the liquid, and, for example, biological binding molecules attached to the surface of microfluidic channel.

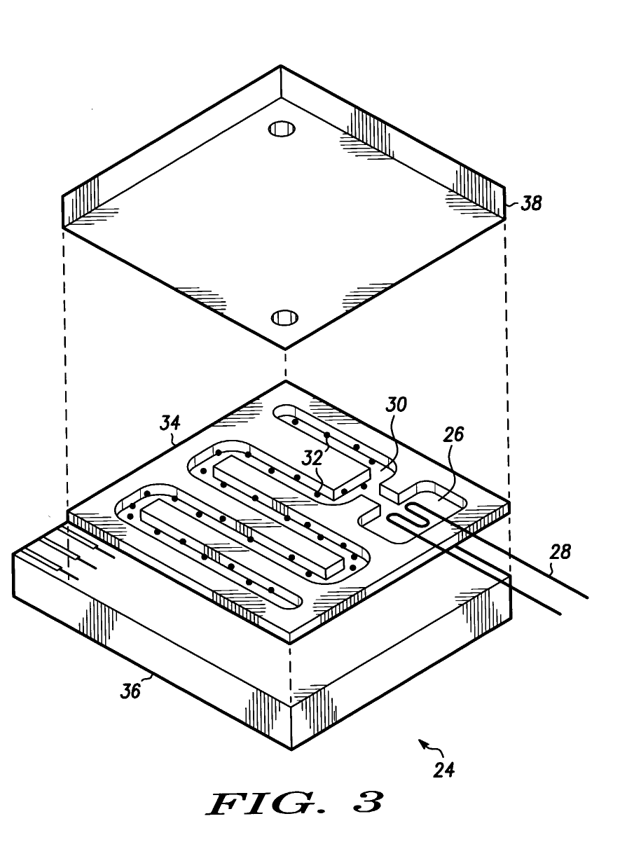

Referring to FIG. 3, an alternative embodiment of the present invention 24 is depicted. Gas pocket 26 is provided with resistive heater 28 to move a liquid sample back and forth within microfluidic channel 30. In some embodiments, a gas vent (not shown) is provided to permit gas to escape or enter as oscillating fluid flow takes place, thereby facilitating fluid flow. Gas pocket 26 and resistive heater 28, may also be referred to herein as an oscillation pump. The skilled artisan will appreciate that oscillation flow is provided by, but not limited to, the oscillation pump. For example, and without limitation, the invention includes such things as diaphragm pumps, electroosmotic, electrohydrodynamic, or electrokinetic pumps, and off chip pumps. Preferably, microfluidic channel 30 has array of biological binding molecules 32 contained therein. Preferably the array of biological molecules 32 is in contact with an array of microelectrodes, which are individually addressable (this type of array is discussed more thoroughly above). Resistive heater 28 is in thermal contact with gas pocket 26 (preferably air). Gas pocket 26 is fluidly connected to microfluidic channel 30. Heating resistive heater 28 causes expansion within gas cavity 26, thereby causing the liquid sample within microfluidic channel 30 to move away from gas pocket 26. Turning off resistive heater 28, thereby cooling the gas within gas pocket 26, results in the fluid within microfluidic channel 30 moving towards gas pocket 26. Thus, expansion and contracting of gas pocket 26 (for example, and not by way of limitation, by heating/cooling of resistive heater 28) results in oscillation of the liquid sample within microfluidic channel 30.

It is believed (again without wishing to be bound by any particular theory) that “target focusing” occurs in a channel array (observed experimentally), because the targets within the solution are forced to pass by each biological binding molecule within the array along the microfluidic channel, and as a result each biological binding molecule “sees” more of the targets within the target solution in the y-direction, as compared to if the target solution were in a much wider channel or a chamber. The x-direction being the flow direction, y-direction being horizontally perpendicular to the flow direction, and z-direction being vertically perpendicular to the flow direction. Thus, constricting the target solution along a channel ensures that much more of the target molecules come in contact with the array probes disposed along the x-direction than would take place in a bulk or more dispersed chamber, because it ensures that virtually the entire liquid sample contacts each of the probes in the array. In a more dispersed reaction chamber where the probes are spatially dispersed relative to the liquid sample, the target molecules will therefore not “see” each of the probes at a distance that permits interaction of the target and the probe molecules. The present invention, by providing the oscillation flow increases exposure of the target molecules to the probes by forcing the entire liquid sample to repeatedly flow across each of the probe molecules in the array.

Additionally, and again not wishing to be bound by any particular theory, it is believed that providing a serpentine or chaotic flow path increases the mixing of the fluid sample in the z-direction, which phenomenon is thoroughly discussed by Liu et al., J. of Microelectromechanical Sys., 9(2):190-197 (June 2000). It is believed, without wishing to be bound by any particular theory, that the combination of these two effects gives rise to accelerated target-biological binding molecule interaction over prior art systems.

As outlined herein, in preferred embodiments, the microfluidic cavity houses an array of biological binding molecules; that is, the microfluidic device comprises a detection module comprising an array, as well as mixing components, as outlined above. As used herein, “microarray of biological binding molecules”, “array of capture binding ligands”, “microarray”, “array” or grammatical equivalents thereof mean a plurality of capture binding ligands, preferably nucleic acids, in an array format; the size of the array will depend on the composition and end use of the array. Most of the discussion herein is directed to the use of nucleic acid arrays with attached nucleic acid probes, but this is not meant to limit the scope of the invention, as other types of biological binding molecules (proteins, etc.) can be used, as is further discussed below. “Array” in this context generally refers to an ordered spatial arrangement, particularly an arrangement of immobilized biomolecules or polymeric anchoring structures. “Addressable array” refers to an array wherein the individual elements have precisely defined X and Y coordinates, so that a given element at a particular position in the array can be identified. By “capture probe”, “binding ligand” or “binding species” herein is meant a compound that is used to probe for the presence of the target analyte, that will bind to the target analyte. Generally, the capture binding ligand allows the attachment of a target analyte to a detection surface, for the purposes of detection. As is more fully outlined below, attachment of the target analyte to the capture binding ligand may be direct (i.e. the target analyte binds to the capture binding ligand) or indirect (one or more capture extender ligands may be used).

In a preferred embodiment, the binding is specific, and the binding ligand is part of a binding pair. By “specifically bind” herein is meant that the ligand binds the analyte, with specificity sufficient to differentiate between the analyte and other components or contaminants of the test sample. However, as will be appreciated by those in the art, it will be possible to detect analytes using binding that is not highly specific; for example, the systems may use different binding ligands, for example an array of different ligands, and detection of any particular analyte is via its “signature” of binding to a panel of binding ligands, similar to the manner in which “electronic noses” work. The binding should be sufficient to allow the analyte to remain bound under the conditions of the assay, including wash steps to remove non-specific binding. In some embodiments, for example in the detection of certain biomolecules, the binding constants of the analyte to the binding ligand will be at least about 10⁻⁴ to 10⁻⁶ M^{31 1}, with at least about 10⁻⁵ to 10⁻⁹ being preferred and at least about 10⁻⁷ to 10⁻⁹ M⁻¹ being particularly preferred.

As will be appreciated by those in the art, the composition of the binding ligand will depend on the composition of the target analyte. Binding ligands to a wide variety of analytes are known or can be readily found using known techniques. For example, when the analyte is a single-stranded nucleic acid, the binding ligand is generally a substantially complementary nucleic acid. Alternatively, as is generally described in U.S. Pat. Nos. 5,270,163, 5,475,096, 5,567,588, 5,595,877, 5,637,459, 5,683,867, 5,705,337, and related patents, hereby incorporated by reference, nucleic acid “aptamers” can be developed for binding to virtually any target analyte. Similarly the analyte may be a nucleic acid binding protein and the capture binding ligand is either a single-stranded or double-stranded nucleic acid; alternatively, the binding ligand may be a nucleic acid binding protein when the analyte is a single or double-stranded nucleic acid. When the analyte is a protein, the binding ligands include proteins (particularly including antibodies or fragments thereof (FAbs, etc.)), small molecules, or aptamers, described above. Preferred binding ligand proteins include peptides. For example, when the analyte is an enzyme, suitable binding ligands include substrates, inhibitors, and other proteins that bind the enzyme, i.e. components of a multi-enzyme (or protein) complex. As will be appreciated by those in the art, any two molecules that will associate, preferably specifically, may be used, either as the analyte or the binding ligand. Suitable analyte/binding ligand pairs include, but are not limited to, antibodies/antigens, receptors/ligand, proteins/nucleic acids; nucleic acids/nucleic acids, enzymes/substrates and/or inhibitors, carbohydrates (including glycoproteins and glycolipids)/lectins, carbohydrates and other binding partners, proteins/proteins; and protein/small molecules. These may be wild-type or derivative sequences. In a preferred embodiment, the binding ligands are portions (particularly the extracellular portions) of cell surface receptors that are known to multimerize, such as the growth hormone receptor, glucose transporters (particularly GLUT4 receptor), transferrin receptor, epidermal growth factor receptor, low density lipoprotein receptor, high density lipoprotein receptor, leptin receptor, interleukin receptors including IL-1, IL-2, IL-3, IL-4, IL-5, IL-6, IL-7, IL-8, IL-9, IL-11, IL-12, IL-13, IL-15 and IL-17 receptors, VEGF receptor, PDGF receptor, EPO receptor, TPO receptor, ciliary neurotrophic factor receptor, prolactin receptor, and T-cell receptors. Similarly, there is a wide body of literature relating to the development of binding partners based on combinatorial chemistry methods.

In a preferred embodiment, the target analytes are nucleic acids and the capture binding ligands are nucleic acid probes (generally referred to herein as “capture probes”). Probes of the present invention are designed to be complementary to a target sequence (either the target sequence of the sample or to other probe sequences), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

Nucleic acids arrays are known in the art, and can be classified in a number of ways; both ordered arrays (e.g., the ability to resolve chemistries at discrete sites), and random arrays are included. Ordered arrays include, but are not limited to, those made using photolithography techniques (Affymetrix GeneChip™), spotting techniques (Synteni and others), printing techniques (Hewlett Packard and Rosetta), and three dimensional “gel pad” arrays, etc. The size of the array can vary; with arrays containing from about 2 different biological binding molecules to many millions can be made, with very large arrays being possible. Generally, the array will comprise from two to as many as 100,000, with from about 400 to about 1000 being the most preferred, and about 10,000 being especially preferred. Arrays can also be classified as “addressable”, which means that the individual elements of the array have precisely defined x and y coordinates, so that a given array element can be pinpointed.

The method of attachment of the capture binding ligands to the detection surface can be done in a variety of ways, depending on the composition of the capture binding ligand and the composition of the detection surface. Examples of constructing an array include, without limitation, photolithography techniques, spotting, and bead arrays. Additionally, the biological binding molecules may be attached to a linker moiety or entrapped within a matrix of linker moieties, which moieties are attached to the substrate surface or an electrode on the substrate. Both direct attachment (e.g. the capture binding ligand such as a nucleic acid probe is directly attached to a conductive polymer layer, gel pad layer, glass substrate, etc.), and indirect attachment, using an attachment linker, can be done. In general, both ways utilize functional groups on the capture binding ligands, the attachment linker, and the detection surface for attachment. Preferred functional groups for attachment are amino groups, carboxy groups, oxo groups and thiol groups. These functional groups can then be attached, either directly or indirectly through the use of a linker, sometimes depicted herein as “Z”. Linkers are well known in the art; for example, homo-or hetero-bifunctional linkers as are well known (see 1994 Pierce Chemical Company catalog, technical section on cross-linkers, pages 155-200, incorporated herein by reference). Preferred modifications to the target analytes useful in the practice of the invention include but are not limited to —OH, —NH₂, —SH, —COOR (where R═H, lower (C₁₋₁₂) alkyl, aryl, heterocyclic alkyl or aryl, or a metal ion), —CN, or —CHO. Immobilization of such derivatized probes is accomplished by direct attaching of the probe molecules on the detection surface through a functional group such —OH, —SH, —NH₂.

Alternatively, probe molecules can be efficiently immobilized on the detection surface through an intermediate species, termed a “spacer.” In these embodiments, the surface of the detection surface is first modified with an intermediate species that carries functional groups such as hydroxyl (—OH), amino (—NH₂), thiol (—SH), carboxyl ester (—COOR, where R═H, lower (C₁₋₁₂) alkyl, aryl, heterocyclic alkyl or aryl, or a metal ion), nitrile (—CN), or aldehylde (—CHO), which can react with the probe molecules functionalized with complementary members of the aforementioned anchoring groups.

In some embodiments of the present invention, the linker moieties of the apparatus are composed of materials including, but not limited to, polyacrylamide gel, agarose gel, polyethylene glycol, cellulose gel, or sol gels. The oligonucleotide probes may be bound to the surface of a continuous layer of the hydrogel, to an array of gel pads or spotted onto a continuous gel layer, or embedded therein. Preferred embodiments are described in WO 01/54814, hereby expressly incorporated by reference. For hydrogel-based arrays using polyacrylamide, biomolecules (such as oligonucleotides, peptides, polypeptides, or proteins) are covalently attached by forming an amide, ester or disulfide bond between the biomolecule and a derivatized polymer comprising the cognate chemical group. Covalent attachment of the biomolecule to the polymer is usually performed after polymerization and chemical cross-linking of the polymer is completed.

Alternatively, oligonucleotides bearing 5′-terminal acrylamide modifications can be used that efficiently copolymerize with acrylamide monomers to form DNA-containing polyacrylamide copolymers (Rehman et al., 1999, Nucleic Acids Research 27: 649-655). Using this approach, stable probecontaining layers can be fabricated on substrates (e.g., microtiter plates and silanized glass) having exposed acrylic groups. This approach has made available the commercially marketed Acrydite™ capture probes (available from Mosaic Technologies, Boston, Mass.). The Acrydite™ moiety is a phosporamidite that contains an ethylene group capable of free-radical copolymerization with acrylamide, and which can be used in standard DNA synthesizers to introduce copolymerizable groups at the 5′ terminus of any oligonucleotide probe.

In alternative embodiments of the present invention, the linker moieties, comprise a conjugated polymer or copolymer film. Such conjugated polymer or copolymer film is composed of materials including, but not limited to, polypyrrole, polythiphene, polyaniline, polyfuran, polypyridine, polycarbazole, polyphenylene, poly(phenylenvinylene), polyfluorene, or polyindole, or their derivatives, their copolymers, and combinations thereof. In a preferred embodiment, the linker moiety is a conductive polymer matrix that has the probe molecules non-covalently immobilized within the polymer matrix, covalently immobilized within the polymer matrix, or covalently attached to the surface of the polymer matrix. The conductive polymers are in-turn in contact with an electrode on the substrate surface, thereby forming an array of electrodes with probe molecules in contact therewith via the conductive polymer. Conductive polymers include polymers that undergo single or multielectron oxidation or reduction reaction in an electrochemical cell. The conductive polymers can be prepared in a film on an electrode surface by electrochemical polymerization of the corresponding monomers, using conventional electrochemical methods, such as and without limitation cyclic voltammetry, or constant potential deposition.

In this embodiment, when the binding ligand is a nucleic acid, preferred compositions and techniques are outlined in U.S. Pat. Nos. 5,591,578; 5,824,473; 5,705,348; 5,780,234 and 5,770,369; U.S. Ser. Nos. 08/873,598 08/911,589; WO 98/20162; WO98/12430; WO98/57158; WO 00/16089) WO99/57317; WO99/67425; WO00/24941; PCT US00/10903; WO00/38836; WO99/37819; WO99/57319 and PCTUS00/20476; and related materials, all of which are expressly incorporated by reference in their entirety. Preferred embodiments utilize arrays of microelectrodes or hydrogel arrays as are known in the art and disclosed, for example, in U.S. Ser. Nos. 09/458,553; 09/458,501; 09/572,187; 09/495,992; 09/344,217; WO00/31148; 09/439,889; 09/438,209; 09/344,620; 09/478,727; PCTUS00/17422; WO 98/20162; WO 98/12430; WO 98/57158; WO 99/57317; WO 99/67425; PCT 00/19889; and WO 99/57319, all of which are incorporated herein by reference in the entirety for all purposes.

Alternatively, the linker moieties may be formed using electrochemical techniques. For example, cyclic voltammetry of pyrrole, 3-acetateN-hydroxysuaccinimido pyrrole and PBS buffer, in the presence of probe molecules, forms a polypyrrole/probe film on a microelectrode surface. The probe molecules are embedded or fixed within the film. The skilled artisan will readily recognize that virtually any method of attaching biological binding molecules to the surface of the substrate, whether it be directly, through a linker, embedded in a linker matrix, or in contact with an array of electrodes directly or through linkers, will fall within the scope of the present invention.

The solid substrate can be made of a wide variety of materials as outlined herein and can be configured in a large number of ways, as is discussed herein and will be apparent to one of skill in the art. In addition, a single device may comprise more than one substrate; for example, there may be a “sample treatment” cassette that interfaces with a separate “detection” cassette; a raw sample is added to the sample treatment cassette and is manipulated to prepare the sample for detection (e.g., cell lysis, PCR amplification; rolling circle amplification; ligase chain reaction (LCR), strand displacement amplification (SDA), and nucleic acid sequence based amplification (NASBA)), which is removed from the sample treatment cassette and added to the detection cassette. There may be an additional functional cassette into which the device fits; for example, a heating element which is placed in contact with the sample cassette to effect reactions such as PCR. In some cases, a portion of the substrate may be removable; for example, the sample cassette may have a detachable detection cassette, such that the entire sample cassette is not contacted with the detection apparatus. See for example U.S. Pat. No. 5,603,351, PCT US96/17116, and PCT US00/33499, incorporated herein by reference in the entirety for all purposes.

As outlined herein, capture binding ligands comprising oligonucleotide probes are particularly preferred. As is appreciated by those in the art, the length of the probe will vary with the length of the target sequence and the hybridization and wash conditions. Generally, oligonucleotide probes range from about 8 to about 50 nucleotides, with from about 10 to about 30 being preferred and from about 12 to about 25 being especially preferred. In some cases, very long probes may be used, e.g. 50 to 200-300 nucleotides in length.

A variety of hybridization conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Accordingly, the present invention provides mixing components, preferably in a detection module. As will be appreciated by those in the art, a variety of detection methods may be used, including, but not limited to, optical detection (as a result of spectral changes upon changes in redox states), which includes fluorescence, phosphorescence, luminiscence, chemiluminescence, electrochemiluminescence, and refractive index; and electronic detection, including, but not limited to, amperommetry, voltammetry, capacitance and impedence. These methods include time or frequency dependent methods based on AC or DC currents, pulsed methods, lock-in techniques, filtering (high pass, low pass, band pass), and time-resolved techniques including time-resolved fluorescence.

In some embodiments, the detection module is configured to allow for optical detection of target analytes. Binding ligands are immobilized on a detection surface. The detection surface may comprise any surface suitable for the attachment of the binding ligands, and preferably comprises a gel pad, more preferably a polyacrylamide gel pad. Particularly preferred embodiments utilize systems outlined in WO 01/54814, incorporated herein in its entirety. Generally, optical detection of target analytes involve providing a colored or luminescent dye as a ‘label’ on the target analyte. Preferred labels include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue™, Texas Red, 1,1′-[1,3-propanediylbis[(dimethylimino-3, 1-propanediyl]]bis[4-[(3-methyl-2(3H) -benzoxazolylidene)methyl]]-,tetraioide, which is sold under the name YOYO-1, and others described in the 6th Edition of the Molecular Probes Handbook by Richard P. Haugland, hereby expressly incorporated by reference.

After binding, a variety of techniques allow for the detection of radiation emitted by the above labels. These techniques include using fiber optic sensors with nucleic acid probes in solution or attached to the fiber optic. Fluorescence is monitored using a photomultiplier tube or other light detection instrument attached to the fiber optic.

In addition, scanning fluorescence detectors such as the Fluorimager sold by Molecular Dynamics are ideally suited to monitoring the fluorescence of modified nucleic acid molecules arrayed on solid surfaces. The advantage of this system is the large number of electron transfer probes that can be scanned at once using chips covered with thousands of distinct nucleic acid probes.

Further, as is known in the art, photodiodes, confocal microscopes, CCD cameras, or active pixel systems maybe used to image the radiation emitted by fluorescent labels.

As will be appreciated by those in the art, there are a variety of electronic and electrochemical detection techniques that can be used. In some embodiments, (e.g. electrochemical detection), hybridization complexes are formed that comprise a target sequence and a capture probe. The target sequence can comprise an electrochemically active reporter (also referred to herein as an electron transfer moiety (ETM)), such as a transition metal complex, defined below. Alternatively, in “sandwich” formats, the hybridization complex further comprises a label probe, that hybridizes to a domain of the target sequence, and comprises the label.

In a preferred embodiment, the detection technique comprises a “sandwich” assay, as is generally described in U.S. Ser. No. 60/073,011 and in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. Although sandwich assays do not result in the alteration of primers, sandwich assays can be considered signal amplification techniques since multiple signals (i.e. label probes) are bound to a single target, resulting in the amplification of the signal. Sandwich assays are used when the target sequence does not comprise a label; that is, when a secondary probe, comprising labels, is used to generate the signal.

As discussed herein, it should be noted that the sandwich assays can be used for the detection of primary target sequences (e.g. from a patient sample), or as a method to detect the product of an amplification reaction as outlined above; thus for example, any of the newly synthesized strands outlined above, for example using PCR, LCR, NASBA, SDA, etc., may be used as the “target sequence” in a sandwich assay.

In a preferred embodiment, the detection surface comprises at least one detection electrode. The capture probe is covalently attached to the electrode, via an “attachment linker”, using a variety of techniques. By “covalently attached” herein is meant that two moieties are attached by at least one bond, including sigma bonds, pi bonds and coordination bonds. Preferred methods utilize conductive polymers or insulators as is generally described in WO 98/20162 and WO 99/57317, both of which are hereby expressly incorporated herein by reference in their entirety.

In a preferred embodiment, the detection surface comprises at least one detection electrode comprising a self-assembled monolayer. As outlined herein, the efficiency of target analyte binding (for example, oligonucleotide hybridization) may increase when the analyte is at a distance from the detectionlectrode. Similarly, non-specific binding of biomolecules, including the target analytes, to a detection electrode is generally reduced when a monolayer is present. Thus, a monolayer facilitates the maintenance of the analyte away from the electrode surface. In addition, a monolayer serves to keep charged species away from the surface of the electrode. Thus, this layer helps to prevent electrical contact between the electrodes and the ETMs, or between the electrode and charged species within the solvent. Such contact can result in a direct “short circuit” or an indirect short circuit via charged species which may be present in the sample. Accordingly, the monolayer is preferably tightly packed in a uniform layer on the electrode surface, such that a minimum of “holes” exist. The monolayer thus serves as a physical barrier to block solvent accesibility to the detection electrode.

The terms “electron donor moiety”, “electron acceptor moiety”, and “ETMs” (ETMs) or grammatical equivalents herein refers to molecules capable of electron transfer under certain conditions. It is to be understood that electron donor and acceptor capabilities are relative; that is, a molecule which can lose an electron under certain experimental conditions will be able to accept an electron under different experimental conditions. It is to be understood that the number of possible electron donor moieties and electron acceptor moieties is very large, and that one skilled in the art of electron transfer compounds will be able to utilize a number of compounds in the present invention. Preferred ETMs include, but are not limited to, transition metal complexes, organic ETMs, and electrodes.

In a preferred embodiment, the ETMs are transition metal complexes. Transition metals are those whose atoms have a partial or complete d shell of electrons. Suitable transition metals for use in the invention include, but are not limited to, cadmium (Cd), copper (Cu), cobalt (Co), palladium (Pd), zinc (Zn), iron (Fe), ruthenium (Ru), rhodium (Rh), osmium (Os), rhenium (Re), platinium (Pt), scandium (Sc), titanium (Ti), Vanadium (V), chromium (Cr), manganese (Mn), nickel (Ni), Molybdenum (Mo), technetium (Tc), tungsten (W), and iridium (Ir). That is, the first series of transition metals, the platinum metals (Ru, Rh, Pd, Os, Ir and Pt), along with Fe, Re, W, Mo and Tc, are preferred. Particularly preferred are ruthenium, rhenium, osmium, platinium, cobalt and iron.

The transition metals are complexed with a variety of ligands, L, to form suitable transition metal complexes, as is well known in the art. L are the co-ligands, that provide the coordination atoms for the binding of the metal ion. As will be appreciated by those in the art, the number and nature of the co-ligands will depend on the coordination number of the metal ion. Mono-, di- or polydentate co-ligands may be used at any position. Thus, for example, when the metal has a coordination number of six, the L from the terminus of the conductive oligomer, the L contributed from the nucleic acid, and r, add up to six. Thus, when the metal has a coordination number of six, r may range from zero (when all coordination atoms are provided by the other two ligands) to four, when all the co-ligands are monodentate. Thus generally, r will be from 0 to 8, depending on the coordination number of the metal ion and the choice of the other ligands.

In one embodiment, the metal ion has a coordination number of six and both the ligand attached to the conductive oligomer and the ligand attached to the nucleic acid are at least bidentate; that is, r is preferably zero, one (i.e. the remaining co-ligand is bidentate) or two (two monodentate co-ligands are used).

As will be appreciated in the art, the co-ligands can be the same or different. Suitable ligands fall into two categories: ligands which use nitrogen, oxygen, sulfur, carbon or phosphorus atoms (depending on the metal ion) as the coordination atoms (generally referred to in the literature as sigma (σ) donors) and organometallic ligands such as metallocene ligands (generally referred to in the literature as pi (π) donors, and depicted herein as Lₘ). Suitable nitrogen donating ligands are well known in the art and include, but are not limited to, NH₂; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam), EDTA, EGTA and isocyanide. Substituted derivatives, including fused derivatives, may also be used. In some embodiments, porphyrins and substituted derivatives of the porphyrin family may be used. See for example, Comprehensive Coordination Chemistry, Ed. Wilkinson et al., Pergammon Press, 1987, Chapters 13.2 (pp73-98), 21.1 (pp. 813-898) and 21.3 (pp 915-957), all of which are hereby expressly incorporated by reference.

Suitable sigma donating ligands using carbon, oxygen, sulfur and phosphorus are known in the art. For example, suitable sigma carbon donors are found in Cotton and Wilkenson, Advanced Organic Chemistry, 5th Edition, John Wiley & Sons, 1988, hereby incorporated by reference; see page 38, for example. Similarly, suitable oxygen ligands include crown ethers, water and others known in the art. Phosphines and substituted phosphines are also suitable; see page 38 of Cotton and Wilkenson.

The oxygen, sulfur, phosphorus and nitrogen-donating ligands are attached in such a manner as to allow the heteroatoms to serve as coordination atoms.

In a preferred embodiment, organometallic ligands are used. In addition to purely organic compounds for use as redox moieties, and various transition metal coordination complexes with δ-bonded organic ligand with donor atoms as heterocyclic or exocyclic substituents, there is available a wide variety of transition metal organometallic compounds with n-bonded organic ligands (see Advanced Inorganic Chemistry, 5th Ed., Cotton & Wilkinson, John Wiley & Sons, 1988, chapter 26; Organometallics, A Concise Introduction, Elschenbroich et al., 2nd Ed., 1992, VCH; and Comprehensive Organometallic Chemistry II, A Review of the Literature 1982-1994, Abel et al. Ed., Vol. 7, chapters 7, 8, 10 & 11, Pergamon Press, hereby expressly incorporated by reference). Such organometallic ligands include cyclic aromatic compounds such as the cyclopentadienide ion [C₅H₅(−1)] and various ring substituted and ring fused derivatives, such as the indenylide (−1) ion, that yield a class of bis(cyclopentadieyl) metal compounds, (i.e. the metallocenes); see for example Robins et al., J. Am. Chem. Soc. 104:1882-1893 (1982); and Gassman et al., J. Am. Chem. Soc. 108:4228-4229 (1986), incorporated by reference. Of these, ferrocene [(C₅H₅)₂Fe] and its derivatives are prototypical examples which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. Metallocene derivatives of a variety of the first, second and third row transition metals are potential candidates as redox moieties that are covalently attached to either the ribose ring or the nucleoside base of nucleic acid. Other potentially suitable organometallic ligands include cyclic arenes such as benzene, to yield bis(arene)metal compounds and their ring substituted and ring fused derivatives, of which bis(benzene)chromium is a prototypical example, Other acyclic n-bonded ligands such as the allyl(−1) ion, or butadiene yield potentially suitable organometallic compounds, and all such ligands, in conjuction with other n-bonded and δ-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are potential candidate redox moieties in nucleic acid analysis.

When one or more of the co-ligands is an organometallic ligand, the ligand is generally attached via one of the carbon atoms of the organometallic ligand, although attachment may be via other atoms for heterocyclic ligands. Preferred organometallic ligands include metallocene ligands, including substituted derivatives and the metalloceneophanes (see page 1174 of Cotton and Wilkenson, supra). For example, derivatives of metallocene ligands such as methylcyclopentadienyl, with multiple methyl groups being preferred, such as pentamethylcyclopentadienyl, can be used to increase the stability of the metallocene. In a preferred embodiment, only one of the two metallocene ligands of a metallocene are derivatized.

As described herein, any combination of ligands may be used. Preferred combinations include: a) all ligands are nitrogen donating ligands; b) all ligands are organometallic ligands; and c) the ligand at the terminus of the conductive oligomer is a metallocene ligand and the ligand provided by the nucleic acid is a nitrogen donating ligand, with the other ligands, if needed, are either nitrogen donating ligands or metallocene ligands, or a mixture.

In addition to transition metal complexes, other organic electron donors and acceptors may be covalently attached to the nucleic acid for use in the invention. These organic molecules include, but are not limited to, riboflavin, xanthene dyes, azine dyes, acridine orange, N,N′-dimethyl-2,7-diazapyrenium dichloride (DAP²⁺), methylviologen, ethidium bromide, quinones such as N,N′-dimethylanthra(2,1,9-def:6,5, 10-d′ef′)diisoquinoline dichloride (ADIQ²⁺); porphyrins ([meso-tetrakis(N-methyl-x-pyridinium)porphyrin tetrachloride], varlamine blue B hydrochloride, Bindschedler's green; 2,6-dichloroindophenol, 2,6-dibromophenolindophenol; Brilliant crest blue (3-amino-9-dimethyl-amino-10-methylphenoxyazine chloride), methylene blue; Nile blue A (aminoaphthodiethylaminophenoxazine sulfate), indigo-5,5′,7,7′-tetrasulfonic acid, indigo-5,5′,7-trisulfonic acid; phenosafranine, indigo-5-monosulfonic acid; safranine T; bis(dimethylglyoximato)-iron(II) chloride; induline scarlet, neutral red, anthracene, coronene, pyrene, 9-phenylanthracene, rubrene, binaphthyl, DPA, phenothiazene, fluoranthene, phenanthrene, chrysene, 1,8-diphenyl-1,3,5,7-octatetracene, naphthalene, acenaphthalene, perylene, TMPD and analogs and subsitituted derivatives of these compounds.

The choice of the specific ETMs will be influenced by the type of electron transfer detection used, as is generally outlined below. Preferred ETMs are metallocenes, with ferrocene being particularly preferred.

In a preferred embodiment, a plurality of ETMs are used.

The ETMs are attached to nucleic acids, target analytes, or soluble binding ligands as is generally outlined in WO 98/20162, hereby expressly incorporated by reference in its entirety.

Detection of electron transfer is generally initiated electronically, with voltage being preferred. A potential is applied to the assay complex. Precise control and variations in the applied potential can be via a potentiostat and either a three electrode system (one reference, one sample (or working) and one counter electrode) or a two electrode system (one sample and one counter electrode). This allows matching of applied potential to peak potential of the system which depends in part on the choice of ETMs (when reporters are used) and in part on the other system components, the composition and integrity of the monolayer, and what type of reference electrode is used. As described herein, ferrocene is a preferred ETM.

In some embodiments, co-reductants or co-oxidants are used as is generally described in WO00/16089, hereby expressly incorporated by reference.

In one embodiment, the efficient transfer of electrons from the ETM to the electrode results in stereotyped changes in the redox state of the ETM. With many ETMs including the complexes of ruthenium containing bipyridine, pyridine and imidazole rings, these changes in redox state are associated with changes in spectral properties. Significant differences in absorbance are observed between reduced and oxidized states for these molecules. See for example Fabbrizzi et al., Chem. Soc. Rev. 1995 pp197-202). These differences can be monitored using a spectrophotometer or simple photomultiplier tube device.

In this embodiment, possible electron donors and acceptors include all the derivatives listed above for photoactivation or initiation. Preferred electron donors and acceptors have characteristically large spectral changes upon oxidation and reduction resulting in highly sensitive monitoring of electron transfer. Such examples include Ru(NH₃)₄py and Ru(bpy)₂im as preferred examples. It should be understood that only the donor or acceptor that is being monitored by absorbance need have ideal spectral characteristics.

In a preferred embodiment, the electron transfer is detected fluorometrically. Numerous transition metal complexes, including those of ruthenium, have distinct fluorescence properties. Therefore, the change in redox state of the electron donors and electron acceptors attached to the nucleic acid can be monitored very sensitively using fluorescence, for example with Ru(4,7-biphenyl₂-phenanthroline)₃²⁺. The production of this compound can be easily measured using standard fluorescence assay techniques. For example, laser induced fluorescence can be recorded in a standard single cell fluorimeter, a flow through “on-line” fluorimeter (such as those attached to a chromatography system) or a multi-sample “plate-reader” similar to those marketed for 96-well immuno assays.

In a further embodiment, electrochemiluminescence is used as the basis of the electron transfer detection. With some ETMs such as Ru²⁺(bpy)₃, direct luminescence accompanies excited state decay. Changes in this property are associated with nucleic acid hybridization and can be monitored with a simple photomultiplier tube arrangement (see Blackburn, G. F. Clin. Chem. 37: 1534-1539 (1991); and Juris et al., supra.

In a preferred embodiment, electronic detection is used, including amperommetry, voltammetry, capacitance, and impedence. Suitable techniques include, but are not limited to, electrogravimetry; coulometry (including controlled potential coulometry and constant current coulometry); voltametry (cyclic voltametry, pulse voltametry (normal pulse voltametry, square wave voltametry, differential pulse voltametry, Osteryoung square wave voltametry, and coulostatic pulse techniques); stripping analysis (aniodic stripping analysis, cathiodic stripping analysis, square wave stripping voltammetry); conductance measurements (electrolytic conductance, direct analysis); time-dependent electrochemical analyses (chronoamperometry, chronopotentiometry, cyclic chronopotentiometry and amperometry, AC polography, chronogalvametry, and chronocoulometry); AC impedance measurement; capacitance measurement; AC voltametry; and photoelectrochemistry.

In a preferred embodiment, monitoring electron transfer is via amperometric detection. This method of detection involves applying a potential (as compared to a separate reference electrode) between the nucleic acid-conjugated electrode and a reference (counter) electrode in the sample containing target genes of interest. Electron transfer of differing efficiencies is induced in samples in the presence or absence of target analyte; that is, the presence or absence of the target analyte, and thus the label probe, can result in different currents.

The device for measuring electron transfer amperometrically involves sensitive current detection and includes a means of controlling the voltage potential, usually a potentiostat. This voltage is optimized with reference to the potential of the electron donating complex on the label probe. Possible electron donating complexes include those previously mentioned with complexes of iron, osmium, platinum, cobalt, rhenium and ruthenium being preferred and complexes of iron being most preferred.

In a preferred embodiment, alternative electron detection modes are utilized. For example, potentiometric (or voltammetric) measurements involve non-faradaic (no net current flow) processes and are utilized traditionally in pH and other ion detectors. Similar sensors are used to monitor electron transfer between the ETM and the electrode. In addition, other properties of insulators (such as resistance) and of conductors (such as conductivity, impedance and capicitance) could be used to monitor electron transfer between ETM and the electrode. Finally, any system that generates a current (such as electron transfer) also generates a small magnetic field, which may be monitored in some embodiments.

In a preferred embodiment, electron transfer is initiated using alternating current (AC) methods. Without being bound by theory, it appears that ETMs, bound to an electrode, generally respond similarly to an AC voltage across a circuit containing resistors and capacitors.

Alternatively, reporterless or labelless systems are used. In this embodiment, two detection electrodes are used to measure changes in capacitance or impedance as a result of target analyte binding. See generally U.S. Ser. No. 09/458,533, filed Dec. 9, 1999 and PCT US00/33497, both of which are expressly incorporated by reference.

In this embodiment, using a labelless system, the surface of the two detection electrodes is covered with a layer of polymer matrix. In these embodiments, probe molecules are attached onto a supporting matrix on the surface of the electrodes using the functional chemistry mentioned above. The polymer matrix is preferably selected to be polypyrrole, polythiophene, polyaniline, polyacrylamide, agarose gel, polyethylene glycol, cellular, sol gels, dendrimers, metallic nanoparticles, carbon nanotubes, and their copolymers. In preferred embodiments, the material comprises a neutral pyrrole matrix. To increase the probe loading capacity, porous matrix such as polyacrylamide, agarose, or sol gels are preferred.

When labels such as ETMs are not used, other initiation/detection systems may be preferred. In this embodiment, molecular interactions between immobilized probe molecules and target molecules in a sample mixture are detected by detecting an electrical signal using AC impedance. In other embodiments, such molecular interactions are detected by detecting an electrical signal using an electrical or electrochemical detection method selected from the group consisting of impedance spectroscopy, cyclic voltammetry, AC voltammetry, pulse voltammetry, square wave voltammetry, AC voltammetry, hydrodynamic modulation voltammetry, conductance, potential step method, potentiometric measurements, amperometric measurements, current step method, other steady-state or transient measurement methods, and combinations thereof.

In one embodiment of the apparatus of the present invention, the means for producing electrical impedance at each test electrode is accomplished using a Model 1260 Impedance/Gain Phase Analyzer with Model 1287 Electrochemical Interface (Solartron Inc., Houston, Tex.). Other electrical impedance measurement means include, but are not limited to, transient methods using AC signal perturbation superimposed upon a DC potential applied to an electrochemical cell such as AC bridge and AC voltammetry. The measurements can be conducted at any particular frequency that specifically produces electrical signal changes that are readily detected or otherwise determined to be advantageous. Such particular frequencies are advantageously determined by scanning frequencies to ascertain the frequency producing, for example, the largest difference in electrical signal. The means for detecting changes in impedance at each test site electrode as a result of molecular interactions between probe and target molecules can be accomplished by using any of the above-described instruments.

In addition to detection modules comprising capture binding ligands and mixing components, the microfluidic devices of the present invention may be configured in a large variety of ways to perform a wide array of applications. Generally, the microfluidic devices of the present invention contain, in addition to the detection module, one or more additional “modules”. By “module”, herein is meant a component or organization of components that enables a certain functionality in the microfluidic device. Modules may be independent and utilized sequentially. Modules may be independent and in fluidic communication with one another by, for example, microchannels. One or more modules may be substantially integrated with one another in the microfluidic device. Examples of a variety of preferred modules are presented below.

“Microfluidic device” as used herein also is intended to include the use of one or more of a variety of components, herein referred to as “modules”, that will be present on any given device depending on its use. These modules include, but are not limited to: sample inlet ports; sample introduction or collection modules; cell handling modules (for example, for cell lysis, cell removal, cell separation or capture, cell growth, etc.); separation modules, for example, for electrophoresis, gel filtration, ion exchange/affinity chromatography (capture and release) etc.; reaction modules for chemical or biological alteration of the sample, including amplification of the target analyte (for example, when the target analyte is nucleic acid, amplification techniques are useful, including, but not limited to polymerase chain reaction (PCR), ligase chain reaction (LCR), strand displacement amplification (SDA), and nucleic acid sequence based amplification (NASBA)), chemical, physical or enzymatic cleavage or alteration of the target analyte, or chemical modification of the target; fluid pumps; fluid valves; thermal modules for heating and cooling (which may be part of other modules, such as reaction modules); storage modules for assay reagents; mixing chambers; and detection modules.

In a preferred embodiment, the devices of the invention include at least one fluid pump. Pumps generally fall into two categories: “on chip” and “off chip”; that is, the pumps (generally electrode based pumps) can be contained within the device itself, or they can be contained on an apparatus into which the device fits, such that alignment occurs of the required flow channels to allow pumping of fluids.

In a preferred embodiment, the pumps are contained on the device itself. These pumps are generally electrode based pumps; that is, the application of electric fields can be used to move both charged particles and bulk solvent, depending on the composition of the sample and of the device. Suitable on chip pumps include, but are not limited to, electroosmotic (EO) pumps and electrohydrodynamic (EHD) pumps; these electrode based pumps have sometimes been referred to in the art as “electrokinetic (EK) pumps”. All of these pumps rely on configurations of electrodes placed along a flow channel to result in the pumping of the fluids comprising the sample components. As is described in the art, the configurations for each of these electrode based pumps are slightly different; for example, the effectiveness of an EHD pump depends on the spacing between the two electrodes, with the closer together they are, the smaller the voltage required to be applied to effect fluid flow. Alternatively, for EO pumps, the spacing between the electrodes should be larger, with up to one-half the length of the channel in which fluids are being moved, since the electrodes are only involved in applying force, and not, as in EHD, in creating charges on which the force will act.

In a preferred embodiment, an electroosmotic pump is used. Electroosmosis (EO) is based on the fact that the surface of many solids, including quartz, glass and others, become variously charged, negatively or positively, in the presence of ionic materials The charged surfaces will attract oppositely charged counterions in aqueous solutions. Applying a voltage results in a migration of the counterions to the oppositely charged electrode, and moves the bulk of the fluid as well. The volume flow rate is proportional to the current, and the volume flow generated in the fluid is also proportional to the applied voltage. Electroosmostic flow is useful for liquids having some conductivity is and generally not applicable for non-polar solvents. EO pumps are described in U.S. Pat. Nos. 4,908,112 and 5,632,876, PCT US95/14586 and WO97/43629, incorporated by reference.

In a preferred embodiment, an electrohydrodynamic (EHD) pump is used. In EHD, electrodes in contact with the fluid transfer charge when a voltage is applied. This charge transfer occurs either by transfer or removal of an electron to or from the fluid, such that liquid flow occurs in the direction from the charging electrode to the oppositely charged electrode. EHD pumps can be used to pump resistive fluids such as non-polar solvents. EHD pumps are described in U.S. Pat. No. 5,632,876, hereby incorporated by reference.

The electrodes of the pumps preferably have a diameter from about 25 microns to about 100 microns, more preferably from about 50 microns to about 75 microns. Preferably, the electrodes protrude from the top of a flow channel to a depth of from about 5% to about 95% of the depth of the channel, with from about 25% to about 50% being preferred. In addition, as described in PCT US95/14586, an electrode-based internal pumping system can be integrated into the liquid distribution system of the devices of the invention with flow-rate control at multiple pump sites and with fewer complex electronics if the pumps are operated by applying pulsed voltages across the electrodes; this gives the additional advantage of ease of integration into high density systems, reductions in the amount of electrolysis that occurs at electrodes, reductions in thermal convection near the electrodes, and the ability to use simpler drivers, and the ability to use both simple and complex pulse wave geometries.

The voltages required to be applied to the electrodes cause fluid flow depends on the geometry of the electrodes and the properties of the fluids to be moved. The flow rate of the fluids is a function of the amplitude of the applied voltage between electrode, the electrode geometry and the fluid properties, which can be easily determined for each fluid. Test voltages used may be up to about 1500 volts, but an operating voltage of about 40 to 300 volts is desirable. An analog driver is generally used to vary the voltage applied to the pump from a DC power source. A transfer function for each fluid is determined experimentally as that applied voltage that produces the desired flow or fluid pressure to the fluid being moved in the channel. However, an analog driver is generally required for each pump along the channel and is suitable an operational amplifier.

In a preferred embodiment, a micromechanical pump is used, either on- or off-chip, as is known in the art.

In a preferred embodiment, an “off-chip” pump is used. For example, the devices of the invention may fit into an apparatus or appliance that has a nesting site for holding the device, that can register the ports (i.e. sample inlet ports, fluid inlet ports, and waste outlet ports) and electrode leads. The apparatus can include pumps that can apply the sample to the device; for example, can force cellcontaining samples into cell lysis modules containing protrusions, to cause cell lysis upon application of sufficient flow pressure. Such pumps are well known in the art.

In a preferred embodiment, on- or off-chip pressure-driven pumps are used. For example, an “air pump” can be used to move fluid. In this embodiment, a chamber of air is incorporated in a device having a heater. When the heater is turned on, the air in the chamber expands according to PV =nRT. Preferably, heaters (as are also described below) are incorporated into the middle of the chip. In some embodiments, more than one heater is incorporated in a chip to create “heater zones”. Air chambers or pockets are located over the heater zones. The air chambers are connected to the reaction chamber via a channel that runs up to the top of the reaction chamber with a valve or a plug blocking it off. When the air is heated, it expands. The resulting build up in pressure forces the valve or plug to move out of the way, thereby forcing the liquid out of the chamber.

Other ways of moving fluid include using a low boiling liquid in place of air. In this embodiment, the low boiling liquid expands when heated and displaces the liquid contained in a chamber. Alternatively, a chemical reaction may be used to move liquid out of a chamber. For example, the chemical reaction used to expand car air bags may be used to move liquid out of the reaction chamber, or other reactions in which gases are generated.

Other types of pressure-based pumps that can be used include syringe driven pumps. These pumps can be actuated either by expanding air behind the syringe or by mechanical means. For example, TiNi alloys, nitinol wire, or “shape memory metals” can be used to mechanically actuate a syringe driven pump. By “TiNi alloys”, “nitinol wire” or “shape memory metals” herein is meant materials that when heated above a certain transition temperature contract (i.e., usually up to 3 to 5% over the original length of the metal), thereby changing shape. Other materials that change shape upon heating include shape memory plastics.

Pumps also may be created using spring loaded pistons. In this embodiment, a spring that can be released is compressed or restrained within the body of the cartridge. For example, wax may be used to hold a spring in its compressed state. Upon heating, the wax is melted, and the spring is released, thereby generating sufficient force to move a piston and displace liquid. Other versions include incorporating materials that change from solids to liquids at a given transition temperature, or moving a mechanical blockade from the spring's pathway. Pumps that utilize PZT driven actuations are also known and may be incorporated in this invention. By “PZT” herein is meant a material comprised of lead, zirconium and titanium which upon application of a voltage undergoes a rearrangement of the crystal lattice and generates a force and a displacement. This so called piezoelectric effect can be used to constrict and expand a pump chamber and result in a net movement of liquid. Other materials like shape memory alloys that under a change in shape upon application of a current such that the temperature of the metal is raised above a certain transition temperature can also be used.

In a preferred embodiment, one or more pumps are used to transport target analytes to a detection module. In another embodiment, one or more pumps are used to contact a module with a sample or an agent, as described below. In other embodiments, pumps are used to agitate a sample or wash contaminant analytes from a concentration module, as described below.

In a preferred embodiment, one or more pumps are used to recirculate the sample within the channels of the device, to allow for increased binding of the target analyte to the capture binding ligand.

In a preferred embodiment, the devices of the invention include at least one fluid valve that can control the flow of fluid into or out of a module of the device, or divert the flow into one or more channels. A variety of valves are known in the art. For example, in one embodiment, the valve may comprise a capillary barrier, as generally described in PCT US97/07880, incorporated by reference. In this embodiment, the channel opens into a larger space designed to favor the formation of an energy minimizing liquid surface such as a meniscus at the opening. Preferably, capillary barriers include a dam that raises the vertical height of the channel immediately before the opening into a larger space such a chamber. In addition, as described in U.S. Pat. No. 5,858,195, incorporated herein by reference, a type of “virtual valve” can be used.

In a preferred embodiment, a chamber in the microfluidic device has one or more valves controlling the flow of fluids into and out of the chamber. The number of valves in the cartridge depends on the number of channels and chambers, and the desired application. Alternatively, the microfluidic device is designed to include one or more loading ports or valves that can be closed off or sealed after the sample is loaded. It is also possible to have multiple loading ports into a single chamber; for example, a first port is used to load sample and a second port is used to add reagents. In these embodiments, the microfluidic device may have a vent. The vent can be configured in a variety of ways. In some embodiments, the vent can be a separate port, optionally with a valve, that leads out of the reaction chamber. Alternatively, the vent may be a loop structure that vents liquid and/or air back into the inlet port.

As will be appreciated by those in the art, a variety of different valves may be used. Valves can be multi cycle or single cycle valves. By “multicycle” valves is meant that the valve can be opened and closed more than once. By “single cycle valves” or “burst valves” or “one time valves” herein is meant a valve that is closed and then opened or opened and then closed but lacks a mechanism for restoring the valve to its original position. Valves may also be check valves, which allow fluid flow in only one direction, or bidirectional valves.

In a preferred embodiment, check valves are used to prevent fluid from going in and out of the reaction chamber during reactions. Generally check valves are used in embodiments where it is desirable to have fluids and/or air flow in one direction, but not the other. For example, when the chamber is filled and thus compressed, air and liquid flow out. Conversely, valves can be used to empty the chamber as well. Types of check valves that can be used include, but are not limited to, duck bill valves (Vernay, www.vernay.com), cantilevers, bubble valves, etc.

In a preferred embodiment, the valve is a cantilever valve. As will be appreciated by those in the art, there are a variety of different types of cantilever valves known in the art. Cantilever valves can also be configured for use in pumping systems as described below. In a preferred embodiment, a cantilever valve comprising a metal is used. In this embodiment, the application of a voltage can either open or close a valve.

In a preferred embodiment, a heat pump is incorporated into the system for opening and closing the cantilever valve. In this embodiment, the check valves are made out of metals such as gold and copper such that the check valve functions as a cantilever when heat is applied. In other embodiments, an actuating force is not used to pull down the valve, rather they have a restraining force that prevents them from going in the other direction.

Similarly, a “thermally actuated” valve that comprises a portion of the microchannel with a flexible membrane filled with air or liquid can be used in conjunction with a heater. The application of heat causes the fluid to expand, blocking the channel.

In other embodiments, piezoelectric (PZT) mixers are used as valves. These can be built out of silicon (obtained from Frauhoffer), plastic (obtained from IMM) or PCB.

Other materials can be used in combination with check valves include materials that can be used to block an inlet or an outlet port. Such materials include wax or other polymeric materials, such as poly(ethylene oxide)-poly(propylene oxide)-poly(ethylene oxide) triblock copolymers (PEO-PPO-PEO) known commercially as Pluronics ( BASF; Pluronic F-127, Sigma) or Synperonic (ICI), that melt for use as membranes or plugs. These materials share the common feature that they can go from a solid to a liquid at a given temperature. These types of systems are used in conjunction with heaters, described below. For example, heat is applied to melt the material, thus “opening” the valve.

In a preferred embodiment, the burst valve is a film of metal or polymer. In a preferred embodiment, a free standing gold film is used, that is constructed using standard techniques as outlined herein, by etching away a support surface. The gold membrane dissolves upon application of a voltage and CI⁻ions. See for example www.mchips.com; Santini, J. T., et al., 1999, Nature, 397:335-338; both of which are incorporated by reference in their entirety.

In a preferred embodiment, a combination of check valves and wax plugs are used. In other embodiments, a combination of check valves and gold membranes are used.

Other means of making a valve include mechanical means. These can frequently be bidirectional valves. For example, a shape memory wire can be attached to a plunger blocking a channel. By applying a current to the wire, the wire contracts and moves the plunger out of the way, thereby opening the channel. Conversely, the plunger can be drawn into the channel to block the channel.

Other mechanical valves include rotary valves. Rotary valves can be configured in a variety of ways. In one embodiment, an external force must be applied for rotation (i.e., a screw driver or stepper motor). Alternatively, a shape memory wire can be used, such that the application of heat or current will shrink the wire to rotate the valve. A complete description of these, and other valves and pumps described above, can be found in WO 01/54813 and PCT US 01/44364, hereby incorporated by reference.

In addition, commercially available valves may be used in to control the flow of liquids from into and out of the various chambers of the present invention. Examples of commercially available valves include, MEMS (micro-electro-mechanical systems) micro valves (www.redwoodmicro.com), TiNi liquid microvalve (TiNi Alloy Company, San Leandro, Calif.), TiNi pneumatic microvalves (TiNi Alloy Company, San Leandro, Calif.), silicon micro valves (Bosch, D., et al., Sensors and Actuators A, 37-38 (1993) 684-692). Commercial/conventional valves also are available from Measurement Specialities, Inc., IC Sensors Division, Milpitas, Calif. (www.msiusa.com/icsensors); Plast-O-Matic Valves, Inc. (www.plastomatic.com), Barworth Inc. (www.barworthinc.com), Mobile Electronics Solution (www.mobileelectronics.net); Specrum Chromatograph (www.lplc.com); all of which are hereby incorporated by reference in their entirety.

Microfluidic devices of the present invention may include a variety of ports, such as inlet or outlet ports, or vents. “Inlet and outlet port” as used herein refers to one or more openings in a microfluidic device suitable for introducing a sample or other fluid into a channel, or removing a sample, waste, or other fluid from the channel. “Vent”, as discussed above, generally refers to an opening in a microfluidic device, or a chamber of the device, for pressure equalization. In one embodiment, the ports are designed for use with conventional pipettes. In another embodiment, multiple inlet ports are provided for the introduction of a variety of fluids, including lysing agents, amplification agents, or sample fluid containing target analytes.

Ports may optionally comprise a seal to prevent or reduce the evaporation of the sample or agents from a chamber. In a preferred embodiment, the seal comprises a gasket, or valve through which a pipette or syringe can be pushed. The gasket or valve can be rubber or silicone or other suitable materials, such as materials containing cellulose.

In another embodiment, the microfluidic device comprises channels or chambers that are substantially open. For example, a chamber or channel having rectangular cross-section may have only three walls. In this embodiment, then, the “inlet port” is the top of the device itself, and may subsequently be sealed with another material comprising the fourth wall of the chamber or channel, or another material, such as mineral oil.

“Microfluidic device” as used herein is further meant to include devices using one or more component to influence or monitor the temperature of a sample, referred to generally as a ‘thermal module’. For example, heaters, including thin-film resistive heating elements, may be provided on- or off-chip. Similarly, coolers, such as heat sinks or heat exchange conduits, may be provided on- or off-chip. Temperature monitoring devices may similarly be incorportated on- or off-chip and are known in the art. The composition and design of heaters, coolers, and temperature monitors will be dictated by the application and the material composition of the microfluidic device.

In one embodiment, heaters, coolers, and temperature monitors are provided to achieve thermal cycling of a chamber to perform PCR.

Suitable thermal modules are described in U.S. Pat. Nos. 5,498,392 and 5,587,128, and WO 97/16561, incorporated by reference, and may comprise electrical resistance heaters, pulsed lasers or other sources of electromagnetic energy directed to the microfluidic device. It should also be noted that when heating elements are used, it may be desirable to have a chamber be relatively shallow, to facilitate heat transfer; see U.S. Pat. No. 5,587,128.

When the devices of the invention include thermal modules, preferred embodiments utilize microfluidic devices having chambers or channels fabricated to have low thermal conductivity in order to minimize thermal crosstalk between adjacent chambers on the microchip, which permits independent thermal control of each chamber or channel.

In certain embodiments, the temperature of a chamber or channel is increased using a thermal module comprising an integrated heater. In preferred embodiments, the integrated heater is a resistive heater, and more preferably a thick film resistive heater plate. Alternatively, chambers or channels can be heated through the use of metal lines integrated beneath the well or surrounding sides of the chambers or channels, more preferably in a coil having one or more loops, in vertical or horizontal orientation. Parallel, variable heating of individual chambers or channels in a microchip array may be accomplished through the use of addressing schemes, preferably a column-and-row or individual electrical addressing scheme, in order to independently control the heat output of the resistive heaters in the vicinity of each chamber or channel.

In certain embodiments, the temperature of the chambers or channels is decreased using a thermal module comprising an integrated cooler. In preferred embodiments, the integrated cooler is a metal via at the bottom of each chamber or channel. In further preferred embodiments, the integrated cooler is a thermo-electric cooler attached to or integrated into the microchip beneath each chamber or channel. Optionally, a metal via is in thermal contact with a metal plate, an array of metal discs or a thermoelectric cooler, each of which functions as a heat sink or an active cooling means. Commercially-available thermoelectric coolers can also be incorporated into the inventive apparatus, because they can be obtained in a wide range of dimensions, including components of a size required for the fabrication of the microfluidic devices of the present invention. In embodiments comprising metal heat sinks encompassing a metal plate or an array of metal discs, the plate or discs are composed of iron, aluminum, or other suitable metal. Parallel, variable cooling of individual chambers or channels in a microfluidic device may be accomplished through the use of addressing schemes, preferably a column-and-row or individual electrical addressing scheme, in order to independently control heat dissipation using cooling elements in the vicinity of each chamber or channel.

In preferred embodiments of the microfluidic devices of the invention, the thermal module includes temperature monitors, to monitor the temperature of the chamber or channel using an integrated resistive thermal detector or a thermocouple. This can be incorporated into the substrate or added later, and is in thermal contact and proximity to the chamber or channel structures of the microfluidic devices of the invention. The resistive thermal detector can be fabricated from a commercially available paste that can be processed in a customized manner for any given design. Such thermocouples are commercially available in sizes of at least 250 microns, including the sheath. In certain alternative embodiments, the temperature of the chambers or channels is monitored using an integrated optical system, for example, an infrared-based system.

In a preferred embodiment, the devices of the invention include a cell handling module. This is of particular use when the sample comprises cells that either contain the target analyte or that must be removed in order to detect the target analyte. Thus, for example, the detection of particular antibodies in blood can require the removal of the blood cells for efficient analysis, or the cells (and/or nucleus) must be lysed prior to detection. In this context, “cells” include eukaryotic and prokaryotic cells, and viral particles that may require treatment prior to analysis, such as the release of nucleic acid from a viral particle prior to detection of target sequences. In addition, cell handling modules may also utilize a downstream means for determining the presence or absence of cells. Suitable cell handling modules include, but are not limited to, cell lysis modules, cell removal modules, and cell separation or capture modules. In addition, as for all the modules of the invention, the cell handling module may be integrated with other modules, or independent and in fluid communication, or capable of being brought into communication, via a channel with at least one other module of the invention.

In a preferred embodiment, the cell handling module includes a cell lysis module. As is known in the art, cells may be lysed in a variety of ways, depending on the cell type. In one embodiment, as described in EP 0 637 998 B1 and U.S. Pat. No. 5,635,358, hereby incorporated by reference, the cell lysis module may comprise cell membrane piercing protrusions that extend from a surface of the cell handling module. As fluid is forced through the device, the cells are ruptured. Similarly, this may be accomplished using sharp edged particles trapped within the cell handling region. Alternatively, the cell lysis module can comprise a region of restricted cross-sectional dimension, which results in cell lysis upon pressure.

In a preferred embodiment, the cell lysis module comprises a cell lysing agent, such as guanidium chloride, chaotropic salts, enzymes such as lysozymes, etc. In some embodiments, for example for blood cells, a simple dilution with water or buffer can result in hypotonic lysis. The lysis agent may be solution form, stored within the cell lysis module or in a storage module and pumped into the lysis module. Alternatively, the lysis agent may be in solid form, that is taken up in solution upon introduction of the sample.

The cell lysis module may also include, either internally or externally, a filtering module for the removal of cellular debris as needed. This filter may be microfabricated between the cell lysis module and the subsequent module to enable the removal of the lysed cell membrane and other cellular debris components; examples of suitable filters are shown in EP 0 637 998 B1, incorporated by reference.

In a preferred embodiment, the cell handling module includes a cell separation or capture module. This embodiment utilizes a cell capture region comprising binding sites capable of reversibly binding a cell surface molecule to enable the selective isolation (or removal) of a particular type of cell from the sample population, for example, white blood cells for the analysis of chromosomal nucleic acid, or subsets of white blood cells. These binding moieties may be immobilized either on the surface of the module or on a particle trapped within the module (i.e. a bead) by physical absorption or by covalent attachment. Suitable binding moieties will depend on the cell type to be isolated or removed, and generally includes antibodies and other binding ligands, such as ligands for cell surface receptors, etc. Thus, a particular cell type may be removed from a sample prior to further handling, or the assay is designed to specifically bind the desired cell type, wash away the non-desirable cell types, followed by either release of the bound cells by the addition of reagents or solvents, physical removal (i.e. higher flow rates or pressures), or even in situ lysis.

Alternatively, a cellular “sieve” can be used to separate cells on the basis of size. This can be done in a variety of ways, including protrusions from the surface that allow size exclusion, a series of narrowing channels, a weir, or a diafiltration type setup.

In a preferred embodiment, the cell handling module includes a cell removal module. This may be used when the sample contains cells that are not required in the assay or are undesirable. Generally, cell removal will be done on the basis of size exclusion as for “sieving”, above, with channels exiting the cell handling module that are too small for the cells.

In a preferred embodiment, the cell handling module includes a cell concentration module. As will be appreciated by those in the art, this is done using “sieving” methods, for example to concentrate the cells from a large volume of sample fluid prior to lysis.

In a preferred embodiment, the devices of the invention include a separation module. Separation in this context means that at least one component of the sample is separated from other components of the sample. This can comprise the separation or isolation of the target analyte, or the removal of contaminants that interfere with the analysis of the target analyte, depending on the assay.

In a preferred embodiment, the separation module includes chromatographic-type separation media such as absorptive phase materials, including, but not limited to reverse phase materials (e.g. C₈ or C₁₈, coated particles, etc.), ion-exchange materials, affinity chromatography materials such as binding ligands, etc. See U.S. Pat. No. 5,770,029, herein incorporated by reference.

In a preferred embodiment, the separation module utilizes binding ligands, as is generally outlined herein for cell separation or analyte detection. In this embodiment, binding ligands are immobilized (again, either by physical absorption or covalent attachment, described below) within the separation module (again, either on the internal surface of the module, on a particle such as a bead, filament or capillary trapped within the module, for example through the use of a frit). Suitable binding moieties will depend on the sample component to be isolated or removed. By “binding ligand” or grammatical equivalents herein is meant a compound that is used to bind a component of the sample, either a contaminant (for removal) or the target analyte (for enrichment). In some embodiments, as outlined below, the binding ligand is used to probe for the presence of the target analyte, and that will bind to the analyte.

In a preferred embodiment, the separation module includes an electrophoresis module, as is generally described in U.S. Pat. Nos. 5,770,029; 5,126,022; 5,631,337; 5,569,364; 5,750,015, and 5,135,627, all of which are hereby incorporated by reference. In electrophoresis, molecules are primarily separated by different electrophoretic mobilities caused by their different molecular size, shape and/or charge. Microcapillary tubes have recently been used for use in microcapillary gel electrophoresis (high performance capillary electrophoresis (HPCE)). One advantage of HPCE is that the heat resulting from the applied electric field is efficiently disappated due to the high surface area, thus allowing fast separation. The electrophoresis module serves to separate sample components by the application of an electric field, with the movement of the sample components being due either to their charge or, depending on the surface chemistry of the microchannel, bulk fluid flow as a result of electroosmotic flow (EOF).

As will be appreciated by those in the art, the electrophoresis module can take on a variety of forms, and generally comprises an electrophoretic microchannel and associated electrodes to apply an electric field to the electrophoretic microchannel. Waste fluid outlets and fluid reservoirs are present as required.

The electrodes comprise pairs of electrodes, either a single pair, or, as described in U.S. Pat. Nos. 5,126,022 and 5,750,015, a plurality of pairs. Single pairs generally have one electrode at each end of the electrophoretic pathway. Multiple electrode pairs may be used to precisely control the movement of sample components, such that the sample components may be continuously subjected to a plurality of electric fields either simultaneously or sequentially.

In a preferred embodiment, electrophoretic gel media may also be used. By varying the pore size of the media, employing two or more gel media of different porosity, and/or providing a pore size gradient, separation of sample components can be maximized. Gel media for separation based on size are known, and include, but are not limited to, polyacrylamide and agarose. One preferred electrophoretic separation matrix is described in U.S. Pat. No. 5,135,627, hereby incorporated by reference, that describes the use of “mosaic matrix”, formed by polymerizing a dispersion of microdomains (“dispersoids”) and a polymeric matrix. This allows enhanced separation of target analytes, particularly nucleic acids. Similarly, U.S. Pat. No. 5,569,364, hereby incorporated by reference, describes separation media for electrophoresis comprising submicron to above-micron sized cross-linked gel particles that find use in microfluidic systems. U.S. Pat. No. 5,631,337, hereby incorporated by reference, describes the use of thermoreversible hydrogels comprising polyacrylamide backbones with N-substituents that serve to provide hydrogen bonding groups for improved electrophoretic separation. See also U.S. Pat. Nos. 5,061,336 and 5,071,531, directed to methods of casting gels in capillary tubes.

In a preferred embodiment, the devices of the invention include a reaction module. This can include either physical, chemical or biological alteration of one or more sample components. Alternatively, it may include a reaction module wherein the target analyte alters a second moiety that can then be detected; for example, if the target analyte is an enzyme, the reaction chamber may comprise an enzyme substrate that upon modification by the target analyte, can then be detected. In this embodiment, the reaction module may contain the necessary reagents, or they may be stored in a storage module and pumped as outlined herein to the reaction module as needed.

In a preferred embodiment, the reaction module includes a chamber for the chemical modification of all or part of the sample. For example, chemical cleavage of sample components (CNBr cleavage of proteins, etc.) or chemical cross-linking can be done. PCT US97/07880, hereby incorporated by reference, lists a large number of possible chemical reactions that can be done in the devices of the invention, including amide formation, acylation, alkylation, reductive amination, Mitsunobu, Diels Alder and Mannich reactions, Suzuki and Stille coupling, chemical labeling, etc. Similarly, U.S. Pat. Nos. 5,616,464 and 5,767,259 describe a variation of LCR that utilizes a “chemical ligation” of sorts. In this embodiment, similar to LCR, a pair of primers are utilized, wherein the first primer is substantially complementary to a first domain of the target and the second primer is substantially complementary to an adjacent second domain of the target (although, as for LCR, if a “gap” exists, a polymerase and dNTPs may be added to “fill in” the gap). Each primer has a portion that acts as a “side chain”0 that does not bind the target sequence and acts as one half of a stem structure that interacts non-covalently through hydrogen bonding, salt bridges, van der Waal's forces, etc. Preferred embodiments utilize substantially complementary nucleic acids as the side chains. Thus, upon hybridization of the primers to the target sequence, the side chains of the primers are brought into spatial proximity, and, if the side chains comprise nucleic acids as well, can also form side chain hybridization complexes. At least one of the side chains of the primers comprises an activatable cross-linking agent, generally covalently attached to the side chain, that upon activation, results in a chemical cross-link or chemical ligation. The activatible group may comprise any moiety that will allow cross-linking of the side chains, and include groups activated chemically, photonically and thermally, with photoactivatable groups being preferred. In some embodiments a single activatable group on one of the side chains is enough to result in cross-linking via interaction to a functional group on the other side chain; in alternate embodiments, activatable groups are required on each side chain. In addition, the reaction chamber may contain chemical moieties for the protection or deprotection of certain functional groups, such as thiols or amines.

In a preferred embodiment, the reaction module includes a chamber for the biological alteration of all or part of the sample. For example, enzymatic processes including nucleic acid amplification, hydrolysis of sample components or the hydrolysis of substrates by a target enzyme, the addition or removal of detectable labels, the addition or removal of phosphate groups, etc.

In a preferred embodiment, the target analyte is a nucleic acid and the biological reaction chamber allows amplification of the target nucleic acid. Suitable amplification techniques include, both target amplification and probe amplification, including, but not limited to, polymerase chain reaction (PCR), ligase chain reaction (LCR), strand displacement amplification (SDA), self-sustained sequence replication (3SR), QB replicase amplification (QBR), repair chain reaction (RCR), cycling probe technology or reaction (CPT or CPR), and nucleic acid sequence based amplification (NASBA). In this embodiment, the reaction reagents generally comprise at least one enzyme (generally polymerase), primers, and nucleoside triphosphates as needed.

General techniques for nucleic acid amplification are discussed below. In most cases, double stranded target nucleic acids are denatured to render them single stranded so as to permit hybridization of the primers and other probes of the invention. A preferred embodiment utilizes a thermal step, generally by raising the temperature of the reaction to about 95° C., although pH changes and other techniques such as the use of extra probes or nucleic acid binding proteins may also be used. Thus, as more fully described above, the reaction chambers of the invention can include thermal modules.

A probe nucleic acid (also referred to herein as a primer nucleic acid) is then contacted to the target sequence to form a hybridization complex. By “primer nucleic acid” herein is meant a probe nucleic acid that will hybridize to some portion, i.e. a domain, of the target sequence. Probes of the present invention are designed to be complementary to a target sequence (either the target sequence of the sample or to other probe sequences, as is described below), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

A variety of hybridization conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Thus, the assays are generally run under stringency conditions which allows formation of the hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The size of the primer nucleic acid may vary, as will be appreciated by those in the art, in general varying from 5 to 500 nucleotides in length, with primers of between 10 and 100 being preferred, between 15 and 50 being particularly preferred, and from 10 to 35 being especially preferred, depending on the use and amplification technique.

In addition, the different amplification techniques may have further requirements of the primers, as is more fully described below.

Once the hybridization complex between the primer and the target sequence has been formed, an enzyme, sometimes termed an “amplification enzyme”, is used to modify the primer. As for all the methods outlined herein, the enzymes may be added at any point during the assay, either prior to, during, or after the addition of the primers. The identification of the enzyme will depend on the amplification technique used, as is more fully outlined below. Similarly, the modification will depend on the amplification technique, as outlined below, although generally the first step of all the reactions herein is an extension of the primer, that is, nucleotides are added to the primer to extend its length.

Once the enzyme has modified the primer to form a modified primer, the hybridization complex is disassociated. Generally, the amplification steps are repeated for a period of time to allow a number of cycles, depending on the number of copies of the original target sequence and the sensitivity of detection, with cycles ranging from 1 to thousands, with from 10 to 100 cycles being preferred and from 20 to 50 cycles being especially preferred.

After a suitable time or amplification, the modified primer can be moved to a detection module and detected.

In a preferred embodiment, the amplification is target amplification. Target amplification involves the amplification (replication) of the target sequence to be detected, such that the number of copies of the target sequence is increased. Suitable target amplification techniques include, but are not limited to, the polymerase chain reaction (PCR), strand displacement amplification (SDA), and nucleic acid sequence based amplification (NASBA).

In a preferred embodiment, the target amplification technique is PCR. The polymerase chain reaction (PCR) is widely used and described, and involve the use of primer extension combined with thermal cycling to amplify a target sequence; see U.S. Pat. Nos. 4,683,195 and 4,683,202, and PCR Essential Data, J. W. Wiley & sons, Ed. C. R. Newton, 1995, all of which are incorporated by reference. In addition, there are a number of variations of PCR which also find use in the invention, including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, “reverse transcriptase PCR” or “RT-PCR”, “biotin capture PCR”, “vectorette PCR”. “panhandle PCR”, and “PCR select cDNA subtration”, among others. In one embodiment, the amplification technique is not PCR.

In general, PCR may be briefly described as follows. A double stranded target nucleic acid is denatured, generally by raising the temperature, and then cooled in the presence of an excess of a PCR primer, which then hybridizes to the first target strand. A DNA polymerase then acts to extend the primer, resulting in the synthesis of a new strand forming a hybridization complex. The sample is then heated again, to disassociate the hybridization complex, and the process is repeated. By using a second PCR primer for the complementary target strand, rapid and exponential amplification occurs. Thus PCR steps are denaturation, annealing and extension. The particulars of PCR are well known, and include the use of a thermostabile polymerase such as Taq I polymerase and thermal cycling.

Accordingly, the PCR reaction requires at least one PCR primer and a polymerase.

In a preferred embodiment, the target amplification technique is SDA. Strand displacement amplification (SDA) is generally described in Walker et al., in Molecular Methods for Virus Detection, Academic Press, Inc., 1995, and U.S. Pat. Nos. 5,455,166 and 5,130,238, all of which are hereby expressly incorporated by reference in their entirety.

In general, SDA may be described as follows. A single stranded target nucleic acid, usually a DNA target sequence, is contacted with an SDA primer. An “SDA primer” generally has a length of 25-100 nucleotides, with SDA primers of approximately 35 nucleotides being preferred. An SDA primer is substantially complementary to a region at the 3′ end of the target sequence, and the primer has a sequence at its 5′ end (outside of the region that is complementary to the target) that is a recognition sequence for a restriction endonuclease, sometimes referred to herein as a “nicking enzyme” or a “nicking endonuclease”, as outlined below. The SDA primer then hybridizes to the target sequence. The SDA reaction mixture also contains a polymerase (an “SDA polymerase”, as outlined below) and a mixture of all four deoxynucleoside-triphosphates (also called deoxynucleotides or dNTPs, i.e. dATP, dTTP, dCTP and dGTP), at least one species of which is a substituted or modified dNTP; thus, the SDA primer is modified, i.e. extended, to form a modified primer, sometimes referred to herein as a “newly synthesized strand”. The substituted dNTP is modified such that it will inhibit cleavage in the strand containing the substituted dNTP but will not inhibit cleavage on the other strand. Examples of suitable substituted dNTPs include, but are not limited, 2′deoxyadenosine 5′-O-(1-thiotriphosphate),5-methyldeoxycytidine 5′-triphosphate, 2′-deoxyuridine 5′-triphosphate, adn 7-deaza-2′-deoxyguanosine 5′-triphosphate. In addition, the substitution of the dNTP may occur after incorporation into a newly synthesized strand; for example, a methylase may be used to add methyl groups to the synthesized strand. In addition, if all the nucleotides are substituted, the polymerase may have 5′-3′ exonuclease activity. However, if less than all the nucleotides are substituted, the polymerase preferably lacks 5′-3′ exonuclease activity.

As will be appreciated by those in the art, the recognition site/endonuclease pair can be any of a wide variety of known combinations. The endonuclease is chosen to cleave a strand either at the recognition site, or either 3′ or 5′ to it, without cleaving the complementary sequence, either because the enzyme only cleaves one strand or because of the incorporation of the substituted nucleotides. Suitable recognition site/endonuclease pairs are well known in the art; suitable endonucleases include, but are not limited to, HincII, HindII, AvaI, Fnu4HI, TthIIII, NcII, BstXI, BamI, etc. A chart depicting suitable enzymes, and their corresponding recognition sites and the modified dNTP to use is found in U.S. Pat. No. 5,455,166, hereby expressly incorporated by reference.

Once nicked, a polymerase (an “SDA polymerase”) is used to extend the newly nicked strand, 5′-3′, thereby creating another newly synthesized strand. The polymerase chosen should be able to intiate 5′-3′ polymerization at a nick site, should also displace the polymerized strand downstream from the nick, and should lack 5′-3′ exonuclease activity (this may be additionally accomplished by the addition of a blocking agent). Thus, suitable polymerases in SDA include, but are not limited to, the Klenow fragment of DNA polymerase I, SEQUENASE 1.0 and SEQUENASE 2.0 (U.S. Biochemical), T5 DNA polymerase and Phi29 DNA polymerase.

Accordingly, the SDA reaction requires, in no particular order, an SDA primer, an SDA polymerase, a nicking endonuclease, and dNTPs, at least one species of which is modified.

In general, SDA does not require thermocycling. The temperature of the reaction is generally set to be high enough to prevent non-specific hybridization but low enough to allow specific hybridization; this is generally from about 37° C. to about 42° C., depending on the enzymes.

In a preferred embodiment, as for most of the amplification techniques described herein, a second amplification reaction can be done using the complementary target sequence, resulting in a substantial increase in amplification during a set period of time. That is, a second primer nucleic acid is hybridized to a second target sequence, that is substantially complementary to the first target sequence, to form a second hybridization complex. The addition of the enzyme, followed by disassociation of the second hybridization complex, results in the generation of a number of newly synthesized second strands.

In a preferred embodiment, the target amplification technique is nucleic acid sequence based amplification (NASBA). NASBA is generally described in U.S. Pat. No. 5,409,818; Sooknanan et al., Nucleic Acid Sequence-Based Amplification, Ch. 12 (pp. 261-285) of Molecular Methods for Virus Detection, Academic Press, 1995; and “Profiting from Gene-based Diagnostics”, CTB International Publishing Inc., N.J., 1996, all of which are incorporated by reference. NASBA is very similar to both TMA and QBR. Transcription mediated amplification (TMA) is generally described in U.S. Pat. Nos. 5,399,491, 5,888,779, 5,705,365, 5,710,029, all of which are incorporated by reference. The main difference between NASBA and TMA is that NASBA utilizes the addition of RNAse H to effect RNA degradation, and TMA relies on inherent RNAse H activity of the reverse transcriptase.

In general, these techniques may be described as follows. A single stranded target nucleic acid, usually an RNA target sequence (sometimes referred to herein as “the first target sequence” or “the first template”), is contacted with a first primer, generally referred to herein as a “NASBA primer” (although “TMA primer” is also suitable). Starting with a DNA target sequence is described below. These primers generally have a length of 25-100 nucleotides, with NASBA primers of approximately 50-75 nucleotides being preferred. The first primer is preferably a DNA primer that has at its 3′ end a sequence that is substantially complementary to the 3′ end of the first template. The first primer also has an RNA polymerase promoter at its 5′ end (or its complement (antisense), depending on the configuration of the system). The first primer is then hybridized to the first template to form a first hybridization complex. The reaction mixture also includes a reverse transcriptase enzyme (an “NASBA reverse transcriptase”) and a mixture of the four dNTPs, such that the first NASBA primer is modified, i.e. extended, to form a modified first primer, comprising a hybridization complex of RNA (the first template) and DNA (the newly synthesized strand).

By “reverse transcriptase” or “RNA-directed DNA polymerase” herein is meant an enzyme capable of synthesizing DNA from a DNA primer and an RNA template. Suitable RNA-directed DNA polymerases include, but are not limited to, avian myloblastosis virus reverse transcriptase (“AMV RT”) and the Moloney murine leukemia virus RT. When the amplification reaction is TMA, the reverse transcriptase enzyme further comprises a RNA degrading activity as outlined below.

In addition to the components listed above, the NASBA reaction also includes an RNA degrading enzyme, also sometimes referred to herein as a ribonuclease, that will hydrolyze RNA of an RNA:DNA hybrid without hydrolyzing single- or double-stranded RNA or DNA. Suitable ribonucleases include, but are not limited to, RNase H from E. coli and calf thymus.

The ribonuclease activity degrades the first RNA template in the hybridization complex, resulting in a disassociation of the hybridization complex leaving a first single stranded newly synthesized DNA strand, sometimes referred to herein as “the second template”.

In addition, the NASBA reaction also includes a second NASBA primer, generally comprising DNA (although as for all the probes herein, including primers, nucleic acid analogs may also be used). This second NASBA primer has a sequence at its 3′ end that is substantially complementary to the 3′ end of the second template, and also contains an antisense sequence for a functional promoter and the antisense sequence of a transcription initiation site. Thus, this primer sequence, when used as a template for synthesis of the third DNA template, contains sufficient information to allow specific and efficient binding of an RNA polymerase and initiation of transcription at the desired site. Preferred embodiments utilizes the antisense promoter and transcription initiation site are that of the T7 RNA polymerase, although other RNA polymerase promoters and initiation sites can be used as well, as outlined below.

The second primer hybridizes to the second template, and a DNA polymerase, also termed a “DNA-directed DNA polymerase”, also present in the reaction, synthesizes a third template (a second newly synthesized DNA strand), resulting in second hybridization complex comprising two newly synthesized DNA strands.

Finally, the inclusion of an RNA polymerase and the required four ribonucleoside triphosphates (ribonucleotides or NTPs) results in the synthesis of an RNA strand (a third newly synthesized strand that is essentially the same as the first template). The RNA polymerase, sometimes referred to herein as a “DNA-directed RNA polymerase”, recognizes the promoter and specifically initiates RNA synthesis at the initiation site. In addition, the RNA polymerase preferably synthesizes several copies of RNA per DNA duplex. Preferred RNA polymerases include, but are not limited to, T7 RNA polymerase, and other bacteriophage RNA polymerases including those of phage T3, phage φII, Salmonella phage sp6, or Pseudomonase phage gh-1.

In some embodiments, TMA and NASBA are used with starting DNA target sequences. In this embodiment, it is necessary to utilize the first primer comprising the RNA polymerase promoter and a DNA polymerase enzyme to generate a double stranded DNA hybrid with the newly synthesized strand comprising the promoter sequence. The hybrid is then denatured and the second primer added.

Accordingly, the NASBA reaction requires, in no particular order, a first NASBA primer, a second NASBA primer comprising an antisense sequence of an RNA polymerase promoter, an RNA polymerase that recognizes the promoter, a reverse transcriptase, a DNA polymerase, an RNA degrading enzyme, NTPs and dNTPs, in addition to the detection components outlined below.

These components result in a single starting RNA template generating a single DNA duplex; however, since this DNA duplex results in the creation of multiple RNA strands, which can then be used to initiate the reaction again, amplification proceeds rapidly.

Accordingly, the TMA reaction requires, in no particular order, a first TMA primer, a second TMA primer comprising an antisense sequence of an RNA polymerase promoter, an RNA polymerase that recognizes the promoter, a reverse transcriptase with RNA degrading activity, a DNA polymerase, NTPs and dNTPs, in addition to the detection components outlined below.

These components result in a single starting RNA template generating a single DNA duplex; however, since this DNA duplex results in the creation of multiple RNA strands, which can then be used to initiate the reaction again, amplification proceeds rapidly.

In a preferred embodiment, the amplification technique is signal amplification. Signal amplification involves the use of limited number of target molecules as templates to either generate multiple signalling probes or allow the use of multiple signalling probes. Signal amplification strategies include LCR, CPT, Invader™, and the use of amplification probes in sandwich assays.

In a preferred embodiment, the signal amplification technique is the oligonucleotide ligation assay (OLA), sometimes referred to as the ligation chain reaction (LCR). The method can be run in two different ways; in a first embodiment, only one strand of a target sequence is used as a template for ligation (OLA); alternatively, both strands may be used (OLA). See generally U.S. Pat. Nos. 5,185,243 and 5,573,907; EP 0 320 308 B1; EP 0 336 731 B1; EP 0 439 182 B1; WO 90/01069; WO 89/12696; and WO 89/09835, and U.S. Ser. Nos. 60/078,102 and 60/073,011, all of which are incorporated by reference.

In a preferred embodiment, the single-stranded target sequence comprises a first target domain and a second target domain, and a first LCR primer and a second LCR primer nucleic acids are added, that are substantially complementary to its respective target domain and thus will hybridize to the target domains. These target domains may be directly adjacent, i.e. contiguous, or separated by a number of nucleotides. If they are non-contiguous, nucleotides are added along with means to join nucleotides, such as a polymerase, that will add the nucleotides to one of the primers. The two LCR primers are then covalently attached, for example using a ligase enzyme such as is known in the art. This forms a first hybridization complex comprising the ligated probe and the target sequence. This hybridization complex is then denatured (disassociated), and the process is repeated to generate a pool of ligated probes.

In a preferred embodiment, LCR is done for two strands of a double-stranded target sequence. The target sequence is denatured, and two sets of probes are added: one set as outlined above for one strand of the target, and a separate set (i.e. third and fourth primer robe nucleic acids) for the other strand of the target. In a preferred embodiment, the first and third probes will hybridize, and the second and fourth probes will hybridize, such that amplification can occur. That is, when the first and second probes have been attached, the ligated probe can now be used as a template, in addition to the second target sequence, for the attachment of the third and fourth probes. Similarly, the ligated third and fourth probes will serve as a template for the attachment of the first and second probes, in addition to the first target strand. In this way, an exponential, rather than just a linear, amplification can occur.

A variation of LCR utilizes a “chemical ligation” of sorts, as is generally outlined in U.S. Pat. Nos. 5,616,464 and 5,767,259, both of which are hereby expressly incorporated by reference in their entirety. In this embodiment, similar to LCR, a pair of primers are utilized, wherein the first primer is substantially complementary to a first domain of the target and the second primer is substantially complementary to an adjacent second domain of the target (although, as for LCR, if a “gap” exists, a polymerase and dNTPs may be added to “fill in” the gap). Each primer has a portion that acts as a “side chain” that does not bind the target sequence and acts one half of a stem structure that interacts non-covalently through hydrogen bonding, salt bridges, van der Waal's forces, etc. Preferred embodiments utilize substantially complementary nucleic acids as the side chains. Thus, upon hybridization of the primers to the target sequence, the side chains of the primers are brought into spatial proximity, and, if the side chains comprise nucleic acids as well, can also form side chain hybridization complexes.

At least one of the side chains of the primers comprises an activatable cross-linking agent, generally covalently attached to the side chain, that upon activation, results in a chemical cross-link or chemical ligation. The activatible group may comprise any moiety that will allow cross-linking of the side chains, and include groups activated chemically, photonically and thermally, with photoactivatable groups being preferred. In some embodiments a single activatable group on one of the side chains is enough to result in cross-linking via interaction to a functional group on the other side chain; in alternate embodiments, activatable groups are required on each side chain.

Once the hybridization complex is formed, and the cross-linking agent has been activated such that the primers have been covalently attached, the reaction is subjected to conditions to allow for the disassocation of the hybridization complex, thus freeing up the target to serve as a template for the next ligation or cross-linking. In this way, signal amplification occurs, and can be detected as outlined herein.

In a preferred embodiment the signal amplification technique is RCA. Rolling-circle amplification is generally described in Baner et al. (1998) Nuc. Acids Res. 26:5073-5078; Barany, F. (1991) Proc. Natl. Acad. Sci. USA 88:189-193; Lizardi et al. (1998) Nat. Genet. 19:225-232; Zhang et al., Gene 211:277 (1998); and Daubendiek et al., Nature Biotech. 15:273 (1997); all of which are incorporated by reference in their entirety.

In general, RCA may be described as follows. First, as is outlined in more detail below, a single RCA probe is hybridized with a target nucleic acid. Each terminus of the probe hybridizes adjacently on the target nucleic acid (or alternatively, there are intervening nucleotides that can be “filled in” using a polymerase and dNTPs, as outlined below) and the OLA assay as described above occurs. When ligated, the probe is circularized while hybridized to the target nucleic acid. Addition of a primer, a polymerase and dNTPs results in extension of the circular probe. However, since the probe has no terminus, the polymerase continues to extend the probe repeatedly. Thus results in amplification of the circular probe. This very large concatamer can be detected intact, as described below, or can be cleaved in a variety of ways to form smaller amplicons for detection as outlined herein.

Accordingly, in an preferred embodiment, a single oligonucleotide is used both for OLA and as the circular template for RCA (referred to herein as a “padlock probe” or a “RCA probe”). That is, each terminus of the oligonucleotide contains sequence complementary to the target nucleic acid and functions as an OLA primer as described above. That is, the first end of the RCA probe is substantially complementary to a first target domain, and the second end of the RCA probe is substantially complementary to a second target domain, adjacent (either directly or indirectly, as outlined herein) to the first domain. Hybridization of the probe to the target nucleic acid results in the formation of a hybridization complex. Ligation of the “primers” (which are the discrete ends of a single oligonucleotide, the RCA probe) results in the formation of a modified hybridization complex containing a circular probe i.e. an RCA template complex. That is, the oligonucleotide is circularized while still hybridized with the target nucleic acid. This serves as a circular template for RCA. Addition of a primer, a polymerase and the required dNTPs to the RCA template complex results in the formation of an amplified product nucleic acid. Following RCA, the amplified product nucleic acid is detected as outlined herein. This can be accomplished in a variety of ways; for example, the polymerase may incorporate labeled nucleotides; a labeled primer may be used, or alternatively, a label probe is used that is substantially complementary to a portion of the RCA probe and comprises at least one label is used.

Accordingly, the present invention provides RCA probes (sometimes referred to herein as “rolling circle probes (RCPs) or “padlock probes” (PPs)). The RCPs may comprise any number of elements, including a first and second ligation sequence, a cleavage site, a priming site, a capture sequence, nucleotide analogs, and a label sequence.

In a preferred embodiment, the RCP comprises first and second ligation sequences. As outlined above for OLA, the ligation sequences are substantially complementary to adjacent domains of the target sequence. The domains may be directly adjacent (i.e. with no intervening bases between the 3′ end of the first and the 5′ of the second) or indirectly adjacent, with from 1 to 100 or more bases in between.

In a preferred embodiment, the RCPs comprise a cleavage site, such that either after or during the rolling circle amplification, the RCP concatamer may be cleaved into amplicons. In some embodiments, this facilitates the detection, since the amplicons are generally smaller and exhibit favorable hybridization kinetics on the surface. As will be appreciated by those in the art, the cleavage site can take on a number of forms, including, but not limited to, the use of restriction sites in the probe, the use of ribozyme sequences, or through the use or incorporation of nucleic acid cleavage moieties.

In a preferred embodiment, the padlock probe contains a restriction site. The restriction endonuclease site allows for cleavage of the long concatamers that are typically the result of RCA into smaller individual units that hybridize either more efficiently or faster to surface bound capture probes. Thus, following RCA (or in some cases, during the reaction), the product nucleic acid is contacted with the appropriate restriction endonuclease. This results in cleavage of the product nucleic acid into smaller fragments. The fragments are then hybridized with the capture probe that is immobilized resulting in a concentration of product fragments onto the detection electrode. Again, as outlined herein, these fragments can be detected in one of two ways: either labelled nucleotides are incorporated during the replication step, for example either as labeled individual dNTPs or through the use of a labeled primer, or an additional label probe is added.

In a preferred embodiment, the restriction site is a single-stranded restriction site chosen such that its complement occurs only once in the RCP.

In a preferred embodiment, the cleavage site is a ribozyme cleavage site as is generally described in Daubendiek et al., Nature Biotech. 15:273 (1997), hereby expressly incorporated by reference. In this embodiment, by using RCPs that encode catalytic RNAs, NTPs and an RNA polymerase, the resulting concatamer can self cleave, ultimately forming monomeric amplicons.

In a preferred embodiment, cleavage is accomplished using DNA cleavage reagents. For example, as is known in the art, there are a number of intercalating moieties that can effect cleavage, for example using light.

In a preferred embodiment, the RCPs do not comprise a cleavage site. Instead, the size of the RCP is designed such that it may hybridize “smoothly” to many capture probes on a surface. Alternatively, the reaction may be cycled such that very long concatamers are not formed.

In a preferred embodiment, the RCPs comprise a priming site, to allow the binding of a DNA polymerase primer. As is known in the art, many DNA polymerases require double stranded nucleic acid and a free terminus to allow nucleic acid synthesis. However, in some cases, for example when RNA polymerases are used, a primer may not be required (see Daubendiek, supra). Similarly, depending on the size and orientation of the target strand, it is possible that a free end of the target sequence can serve as the primer; see Baner et al., supra.

Thus, in a preferred embodiment, the padlock probe also contains a priming site for priming the RCA reaction. That is, each padlock probe comprises a sequence to which a primer nucleic acid hybridizes forming a template for the polymerase. The primer can be found in any portion of the circular probe. In a preferred embodiment, the primer is located at a discrete site in the probe. In this embodiment, the primer site in each distinct padlock probe is identical, although this is not required. Advantages of using primer sites with identical sequences include the ability to use only a single primer oligonucleotide to prime the RCA assay with a plurality of different hybridization complexes. That is, the padlock probe hybridizes uniquely to the target nucleic acid to which it is designed. A single primer hybridizes to all of the unique hybridization complexes forming a priming site for the polymerase. RCA then proceeds from an identical locus within each unique padlock probe of the hybridization complexes.

In an alternative embodiment, the primer site can overlap, encompass, or reside within any of the above-described elements of the padlock probe. That is, the primer can be found, for example, overlapping or within the restriction site or the identifier sequence. In this embodiment, it is necessary that the primer nucleic acid is designed to base pair with the chosen primer site.

In a preferred embodiment, the primer may comprise a covalently attached label.

In a preferred embodiment, the RCPs comprise a capture sequence. A capture sequence, as is outlined herein, is substantially complementary to a capture probe, as outlined herein.

In a preferred embodiment, the RCPs comprise a label sequence; i.e. a sequence that can be used to bind label probes and is substantially complementary to a label probe. In one embodiment, it is possible to use the same label sequence and label probe for all padlock probes on an array; alternatively, each padlock probe can have a different label sequence.

In a preferred embodiment, the RCP/primer sets are designed to allow an additional level of amplification, sometimes referred to as “hyperbranching” or “cascade amplification”. As described in Zhang et al., supra, by using several priming sequences and primers, a first concatamer can serve as the template for additional concatamers. In this embodiment, a polymerase that has high displacement activity is preferably used. In this embodiment, a first antisense primer is used, followed by the use of sense primers, to generate large numbers of concatamers and amplicons, when cleavage is used.

Thus, the invention provides for methods of detecting using RCPs as described herein. Once the ligation sequences of the RCP have hybridized to the target, forming a first hybridization complex, the ends of the RCP are ligated together as outlined above for OLA. The RCP primer is added, if necessary, along with a polymerase and dNTPs (or NTPs, if necessary).

The polymerase can be any polymerase as outlined herein, but is preferably one lacking 3′ exonuclease activity (3′ exo⁻). Examples of suitable polymerase include but are not limited to exonuclease minus DNA Polymerase I large (Klenow) Fragment, Phi29 DNA polymerase, Taq DNA Polymerase and the like. In addition, in some embodiments, a polymerase that will replicate single-stranded DNA (i.e. without a primer forming a double stranded section) can be used.

Thus, in a preferred embodiment the OLA/RCA is performed in solution followed by restriction endonuclease cleavage of the RCA product. The cleaved product is then applied to an array as described herein. The incorporation of an endonuclease site allows the generation of short, easily hybridizable sequences. Furthermore, the unique capture sequence in each rolling circle padlock probe sequence allows diverse sets of nucleic acid sequences to be analyzed in parallel on an array, since each sequence is resolved on the basis of hybridization specificity.

In a preferred embodiment, the polymerase creates more than 100 copies of the circular DNA. In more preferred embodiments the polymerase creates more than 1000 copies of the circular DNA; while in a most preferred embodiment the polymerase creates more than 10,000 copies or more than 50,000 copies of the template.

The RCA as described herein finds use in allowing highly specific and highly sensitive detection of nucleic acid target sequences. In particular, the method finds use in improving the multiplexing ability of DNA arrays and eliminating costly sample or target preparation. As an example, a substantial savings in cost can be realized by directly analyzing genomic DNA on an array, rather than employing an intermediate PCR amplification step. The method finds use in examining genomic DNA and other samples including mRNA.

In addition the RCA finds use in allowing rolling circle amplification products to be easily detected by hybridization to probes in a solid-phase format. An additional advantage of the RCA is that it provides the capability of multiplex analysis so that large numbers of sequences can be analyzed in parallel. By combining the sensitivity of RCA and parallel detection on arrays, many sequences can be analyzed directly from genomic DNA.

In a preferred embodiment, the signal amplification technique is CPT. CPT technology is described in a number of patents and patent applications, including U.S. Pat. Nos. 5,011,769, 5,403,711, 5,660,988, and 4,876,187, and PCT published applications WO 95/05480, WO 95/1416, and WO 95/00667, and U.S. Ser. No. 09/014,304, all of which are expressly incorporated by reference in their entirety.

Generally, CPT may be described as follows. A CPT primer (also sometimes referred to herein as a “scissile primer”), comprises two probe sequences separated by a scissile linkage. The CPT primer is substantially complementary to the target sequence and thus will hybridize to it to form a hybridization complex. The scissile linkage is cleaved, without cleaving the target sequence, resulting in the two probe sequences being separated. The two probe sequences can thus be more easily disassociated from the target, and the reaction can be repeated any number of times. The cleaved primer is then detected as outlined herein.

By “scissile linkage” herein is meant a linkage within the scissile probe that can be cleaved when the probe is part of a hybridization complex, that is, when a double-stranded complex is formed. It is important that the scissile linkage cleave only the scissile probe and not the sequence to which it is hybridized (i.e. either the target sequence or a probe sequence), such that the target sequence may be reused in the reaction for amplification of the signal. As used herein, the scissile linkage, is any connecting chemical structure which joins two probe sequences and which is capable of being selectively cleaved without cleavage of either the probe sequences or the sequence to which the scissile probe is hybridized. The scissile linkage may be a single bond, or a multiple unit sequence.

As will be appreciated by those in the art, a number of possible scissile linkages may be used.

In a preferred embodiment, the scissile linkage comprises RNA. This system, previously described in as outlined above, is based on the fact that certain double-stranded nucleases, particularly ribonucleases, will nick or excise RNA nucleosides from a RNA:DNA hybridization complex. Of particular use in this embodiment is RNAseH, Exo III, and reverse transcriptase.

In one embodiment, the entire scissile probe is made of RNA, the nicking is facilitated especially when carried out with a double-stranded ribonuclease, such as RNAseH or Exo III. RNA probes made entirely of RNA sequences are particularly useful because first, they can be more easily produced enzymatically, and second, they have more cleavage sites which are accessible to nicking or cleaving by a nicking agent, such as the ribonucleases. Thus, scissile probes made entirely of RNA do not rely on a scissile linkage since the scissile linkage is inherent in the probe.

In a preferred embodiment, InvaderTM technology is used. Invader™ technology is based on structure-specific polymerases that cleave nucleic acids in a site-specific manner. Two probes are used: an “invader” probe and a “signaling” probe, that adjacently hybridize to a target sequence with a non-complementary overlap. The enzyme cleaves at the overlap due to its recognition of the “tail”, and releases the “tail”. This can then be detected. The Invader™ technology is described in U.S. Pat. Nos. 5,846,717; 5,614,402; 5,719,028; 5,541,311; and 5,843,669, all of which are hereby incorporated by reference.

Accordingly, the invention provides a first primer, sometimes referred to herein as an “invader primer”, that hybridizes to a first domain of a target sequence, and a second primer, sometimes referred to herein as the signaling primer, that hybridizes to a second domain of the target sequence. The first and second target domains are adjacent. The signaling primer further comprises an overlap sequence, comprising at least one nucleotide, that is perfectly complementary to at least one nucleotide of the first target domain, and a non-complementary “tail” region. The cleavage enzyme recognizes the overlap structure and the noncomplementary tail, and cleaves the tail from the second primer. Suitable cleavage enzymes are described in the Patents outlined above, and include, but are not limited to, 5′ thermostable nucleases from Thermus species, including Thermus aquaticus, Thermus flavus and Thermus thermophilus. The entire reaction is done isothermally at a temperature such that upon cleavage, the invader probe and the cleaved signaling probe come off the target stand, and new primers can bind. In this way large amounts of cleaved signaling probe (i.e. “tails”) are made. The uncleaved signaling probes are removed (for example by binding to a solid support such as a bead, either on the basis of the sequence or through the use of a binding ligand attached to the portion of the signaling probe that hybridizes to the target). The cleaved signalling probes are then detected as outlined herein.

In this way, a number of target molecules are made. As is more fully outlined below, these reactions (that is, the products of these reactions) can be detected in a number of ways, as is generally outlined in U.S. Ser. Nos. 09/458,553; 09/458,501; 09/572,187; 09/495,992; 09/344,217; WO00/31148; 09/439,889; 09/438,209; 09/344,620; PCTUS00/17422; 09/478,727, all of which are expressly incorporated by reference in their entirety.

In addition to the components outlined above for reaction modules, as described in U.S. Pat. No. 5,587,128, the reaction module may comprise a composition, either in solution or adhered to the surface of the reaction module, that prevents the inhibition of an amplification reaction by the composition of the well. For example, the wall surfaces may be coated with a silane, for example using a silanization reagent such as dimethylchlorosilane, or coated with a siliconizing reagent such as Aquasil™ or Surfacil™ (Pierce, Rockford, Ill.), which are organosilanes containing a hydrolyzable group. This hydrolyzable group can hydrolyze in solution to form a silanol that can polymerize and form a tightly bonded film over the surface of the chamber. The coating may also include a blocking agent that can react with the film to further reduce inhibition; suitable blocking agents include amino acid polymers and polymers such as polyvinylpyrrolidone, polyadenylic acid and polymaleimide. Alternatively, for silicon substrates, a silicon oxide film may be provided on the walls, or the reaction chamber can be coated with a relatively inert polymer such as a polyvinylchloride. In addition, it may be desirable to add blocking polynucleotides to occupy any binding sites on the surface of the chamber.

In a preferred embodiment, the biological reaction chamber allows enzymatic cleavage or alteration of the target analyte. For example, restriction endonucleases may be used to cleave target nucleic acids comprising target sequences, for example genomic DNA, into smaller fragments to facilitate either amplification or detection. Alternatively, when the target analyte is a protein, it may be cleaved by a protease. Other types of enzymatic hydrolysis may also be done, depending on the composition of the target analyte. In addition, as outlined herein, the target analyte may comprise an enzyme and the reaction chamber comprises a substrate that is then cleaved to form a detectable product.

In addition, in one embodiment the reaction module includes a chamber for the physical alteration of all or part of the sample, for example for shearing genomic or large nucleic acids, nuclear lysis, ultrasound, etc.

As described herein, there are three general ways that the assays of the invention are run. In a first embodiment, the target analyte is labeled; binding of the target analyte thus provides the label at the surface of the solid support. Alternatively, in a second embodiment, unlabeled target analytes are used, and a “sandwich” format is utilized; in this embodiment, there are at least two binding ligands used per target analyte molecule; a “capture” or “anchor” binding ligand (also referred to herein as a “capture probe”, particularly in reference to a nucleic acid binding ligand) that is attached to the detection surface as described herein, and a soluble binding ligand (frequently referred to herein as a “signaling probe” or “label probe”), that binds independently to the target analyte, and either directly or indirectly comprises at least one label. In a third embodiment, as further outlined herein, none of the compounds comprises a label, and the system relies on changes in electronic properties for detection.

In a preferred embodiment, the devices of the invention comprise liquid handling components, including components for loading and unloading fluids at each station or sets of stations. The liquid handling systems can include robotic systems comprising any number of components. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; holders with cartridges and/or caps; automated lid or cap handlers to remove and replace lids for wells on non-cross contamination plates; tip assemblies for sample distribution with disposable tips; washable tip assemblies for sample distribution; 96 well loading blocks; cooled reagent racks; microtitler plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, highdensity transfers, full-plate serial dilutions, and high capacity operation.

In a preferred embodiment, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In a preferred embodiment, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station.

In a preferred embodiment, thermocycler and thermoregulating systems are used for stabilizing the temperature of heat exchangers such as controlled blocks or platforms to provide accurate temperature control of incubating samples from 0° C. to 100° C.; this is in addition to or in place of the station thermocontrollers.

In a preferred embodiment, interchangeable pipet heads (single or multi-channel ) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, for example when electronic detection is not done, the instrumentation will include a detector, which can be a wide variety of different detectors, depending on the labels and assay. In a preferred embodiment, useful detectors include a microscope(s) with multiple channels of fluorescence; plate readers to provide fluorescent, ultraviolet and visible spectrophotometric detection with single and dual wavelength endpoint and kinetics capability, fluroescence resonance energy transfer (FRET), luminescence, quenching, two-photon excitation, and intensity redistribution; CCD cameras to capture and transform data and images into quantifiable formats; and a computer workstation.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flow cytometry or capillary electrophoresis formats can be used for individual capture of magnetic and other beads, particles, cells, and organisms.

The flexible hardware and software allow instrument adaptability for multiple applications. The software program modules allow creation, modification, and running of methods. The system diagnostic modules allow instrument alignment, correct connections, and motor operations. The customized tools, labware, and liquid, particle, cell and organism transfer patterns allow different applications to be performed. The database allows method and parameter storage. Robotic and computer interfaces allow communication between instruments.

In a preferred embodiment, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

These robotic fluid handling systems can utilize any number of different reagents, including buffers, reagents, samples, washes, assay components such as label probes, etc.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXAMPLES

Fluidic dye experiments (including single-gas pocket and multiple-gas pocket tests) were implemented to visualize and study gas pocket-induced acoustic mixing. Mixing tests were performed in 4-up biochip chambers and e-Sensor™ (Clinical Micro Sensor Inc.) devices. High-density 2-oligo array hybridization was performed to evaluate mixing enhancement and the resulting improvement in efficiency and uniformity over conventional diffusion-based static hybridization.

### Example 1

Fluidic dye experiments were carried out in an optically transparent shallow chamber. The chamber was constructed by sealing a planar piece of polycarbonate layer, which has a cavity machined in the surface, with a plastic (polycarbonate) cover layer using either thermal bonding or double-sided adhesive tape. The chamber is 300 μm deep and 15 mm diameter. The chamber contents are irradiated by sound that comes from a PZT disk (15 mm diameter) bonded (using a super-glue, or other appropriate adhesive) to the external surface of the cavity layer and opposite thereto. The PZT crystal was driven by a Hewlett-Packard functional generator. Visual observations are made from above, using a stereoscope. The chamber is filled half space with DI water and the other half with a red dye solution (a mixture of phenolphthalein and sodium hydroxide solution) that is used to visualize fluid motion and mixing within the chamber. The frequencies employed are around 3 kHz (sinusoidal sound wave) with 5 V peak-to-peak amplitude.

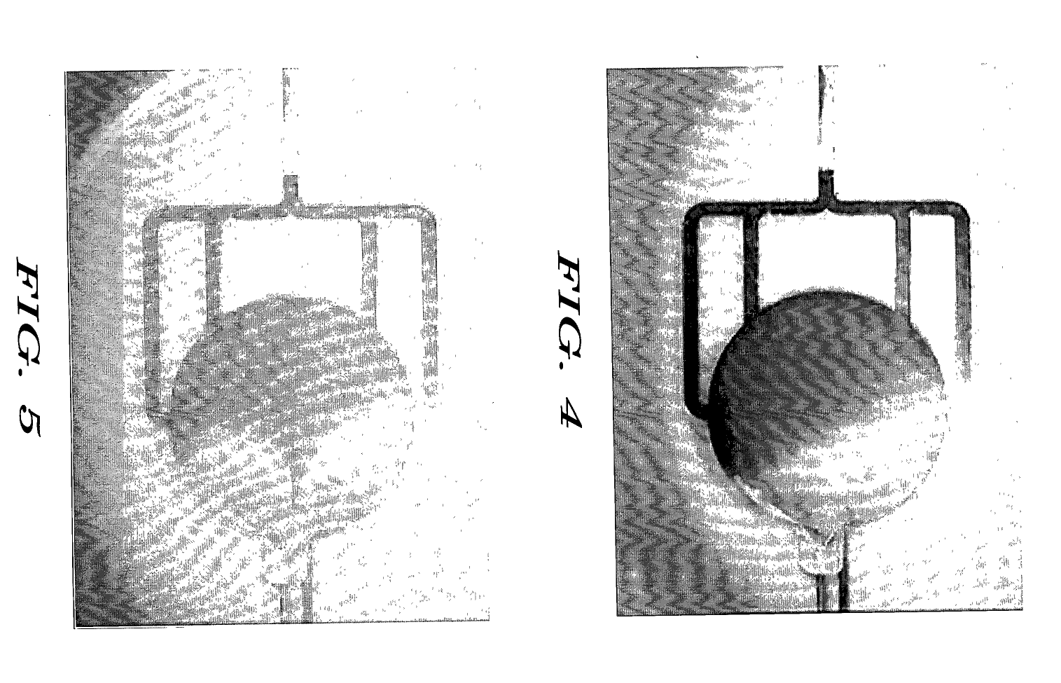

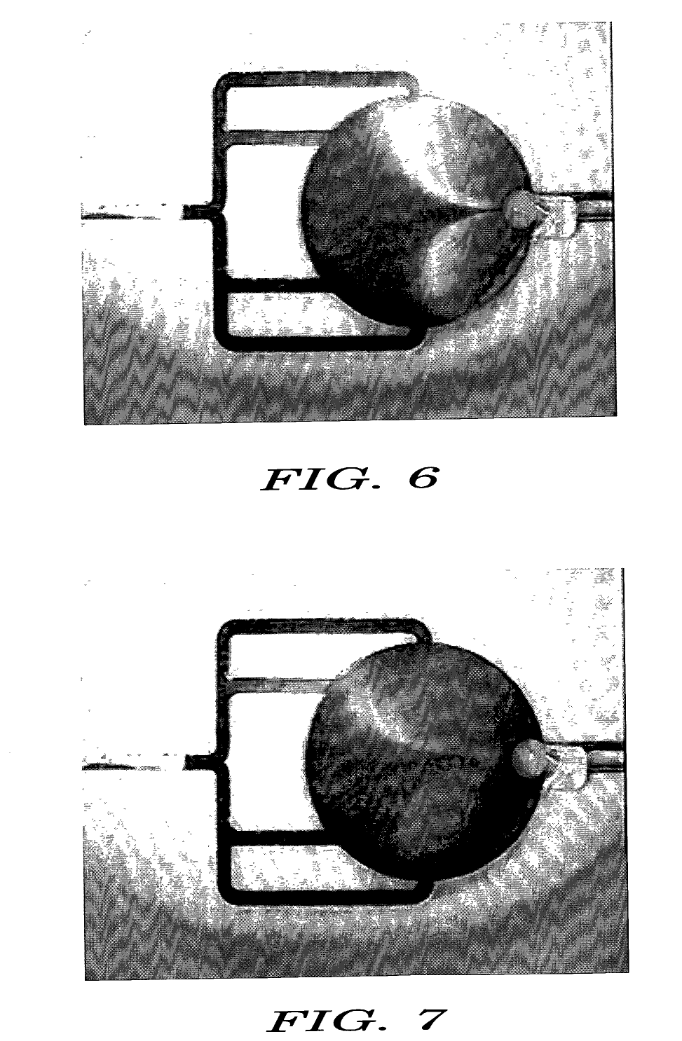

It was found that sonic irradiation caused little motion of the liquid, if gas pockets were excluded from the chamber. However, with a small gas pocket (an approximately 2 mm diameter air bubble), stabilized at the corner of the chamber during the solution filling process, a gross liquid motion was seen to take place (FIG. 4). Churning motion in the liquid was seen at the air-liquid interface. An energetic convection streaming motion (looks like a “tornado” pattern) was observed in the vicinity of the bubble, as shown in FIGS. 5-7.

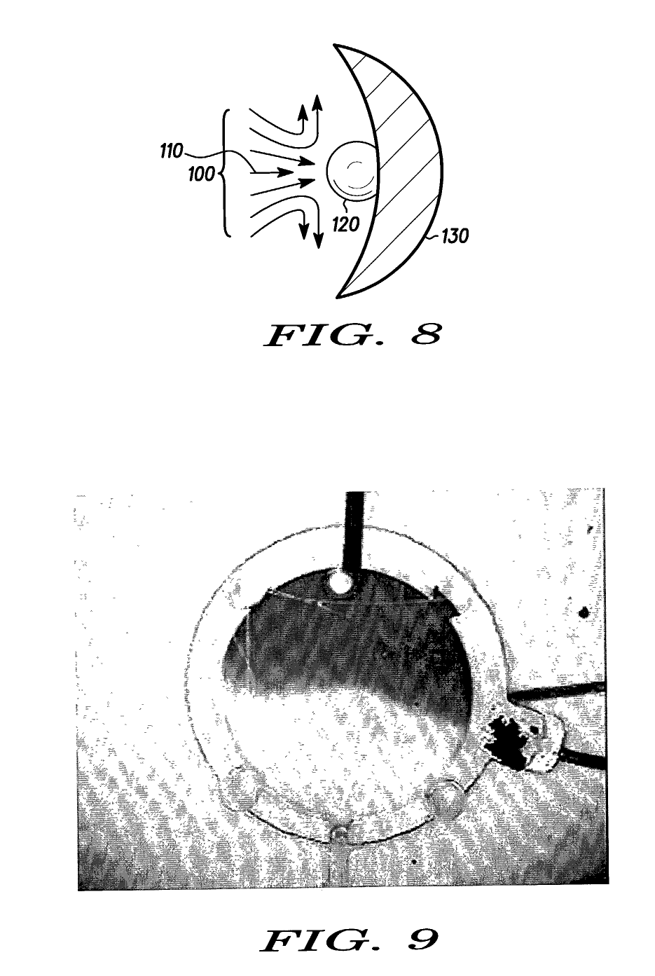

A conceptual sketch of the acoustic streaming pattern around the bubble is illustrated in FIG. 8. The sketch is provided by way of mechanistic example, and is no way intended to limit the invention or the mechanisms by which fluid mixing may occur. The streaming field, schematically indicated by element lines 100, consists of orderly patterns with symmetry about axis 110 perpendicular to the solid wall 130 and extending through the center of bubble 120. Fluid elements move toward the bubble along the axis of symmetry 110. Upon nearing bubble 120, the elements suddenly change direction and are projected outward along the tangential plane to the surface of bubble 120. The speed of the fluid elements is relatively large (estimated to be −5 mm/sec), when focused into a narrow stream, and move toward the bubble surface. The speed then decreases as the elements spread out and leave the bubble region. Flow circulation could also be seen in the liquid.

### Example 2

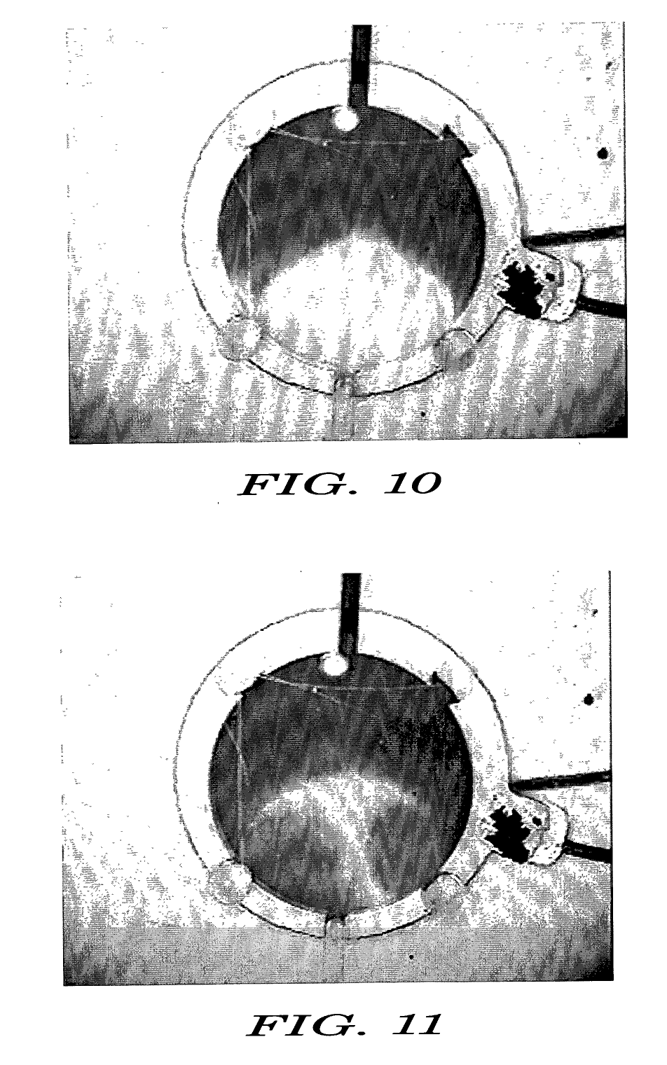

Precisely controlling the bubble size is preferred for achieving repeatable and consistent mixing enhancement effect. Since polycarbonate is a hydrophobic material, air bubbles can be easily trapped in small indentations inside the chamber that is filled with liquid solutions. Thus, polycarbonate was used for this preferred example, however, the skilled artisan will recognize that other materials may be used to construct the microfluidic chamber. The dimension of the pockets defines the size of bubbles. In order to increase the rate and uniformity of mixing, four air bubbles were stabilized in indentations around the chamber (15 mm diameter) as shown in FIGS. 9-12. These indentations with a size of 2 mm diameter and 300 μm depth were micromachined using a Prolight milling machine. The skilled artisan will recognize that other shapes of pockets may be used, and that other methods of forming the indentation may be used without exceeding the scope of the present invention. The PZT transducer, adhered to the chamber, was driven with the same parameters as described in Example 155. Referring to FIGS. 9-12 again, a large amount of gross liquid motion was observed upon application of the acoustic waves. Orderly vortex motions were observed near the individual bubbles. Churning motion in the liquid was seen at the air-liquid interfaces. “Tornado” fluid movements were observed in the vicinity of the bubbles. The time taken to fully mix the whole chamber is approximately 45 sec, almost half of the mixing time as with a single bubble described in Example 1.

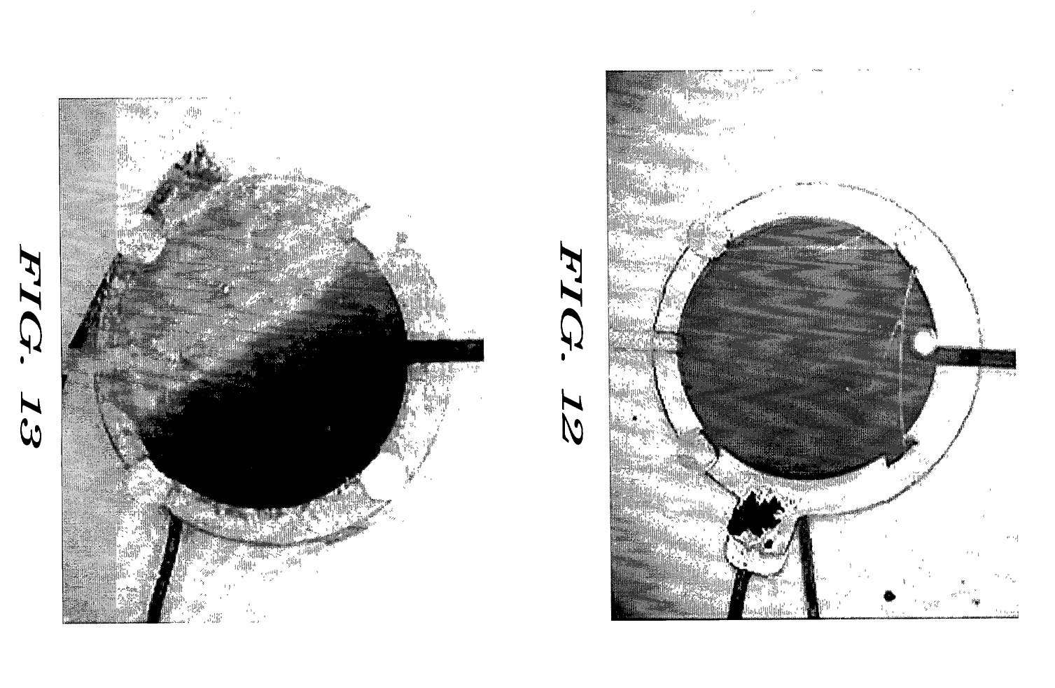

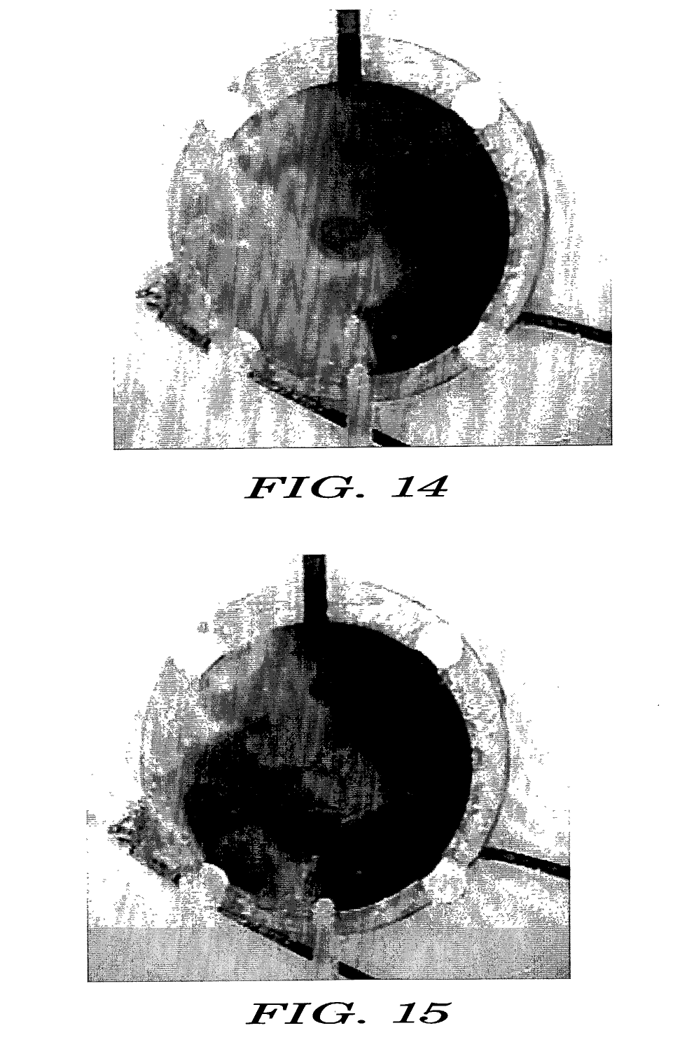

The mixing rate can be further improved by constructing (drilling) a number of air pockets (0.5 mm diameter and 0.5 mm depth) inside the top polycarbonate layer of the chamber, as shown in FIGS. 1-2 and 13-16. Since these top pockets are uniformly distributed above the chamber, the resulting acoustic streaming dominates the mixing in the whole chamber within a few seconds. As shown in FIGS. 13-16, when the PZT was turned on and as the dye moved from one side to another the side, the streams begin to interfere with each other. As streaming continues the mixing becomes faster and eventually completely dominates the picture. Since the top pockets are smaller than the side pockets, the resonant frequency is hence higher for the top pockets to enhance mixing according to Eq. (1), given above. 5.3 kHz was employed here. As a result, the bubble-induced motions around the side pockets are not as violent and chaotic as those around the top pockets.

### Example 3

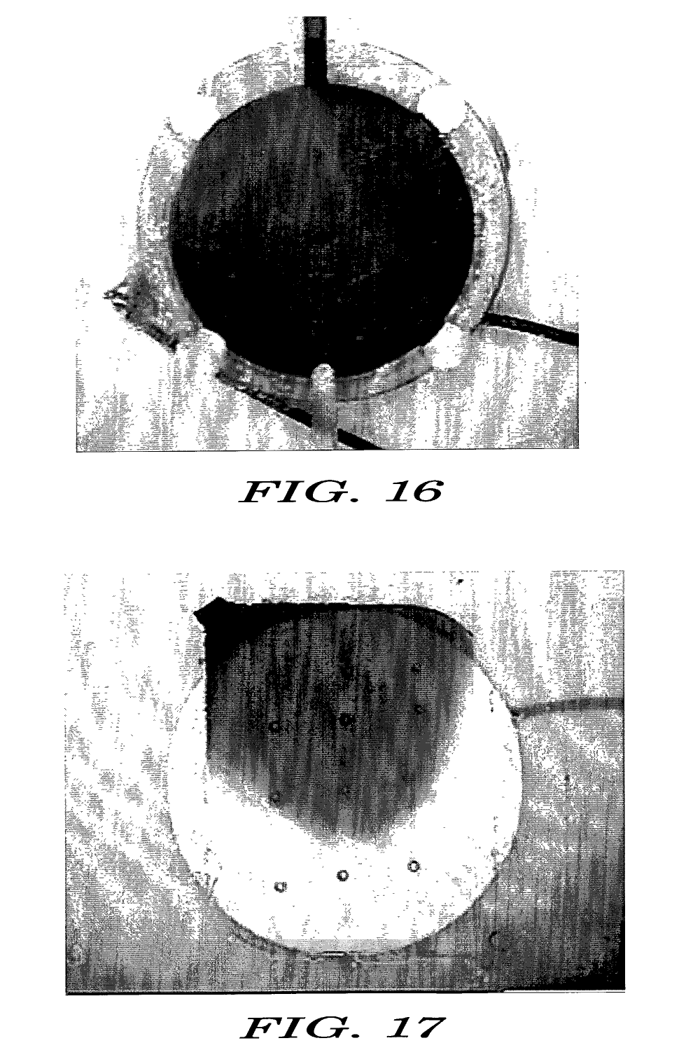

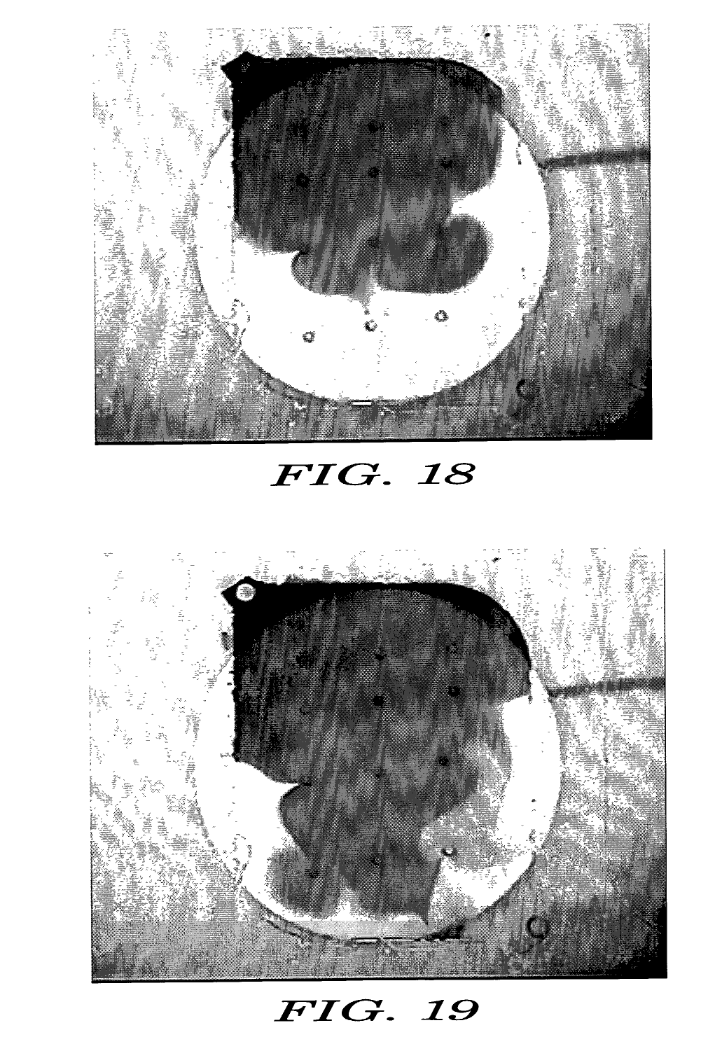

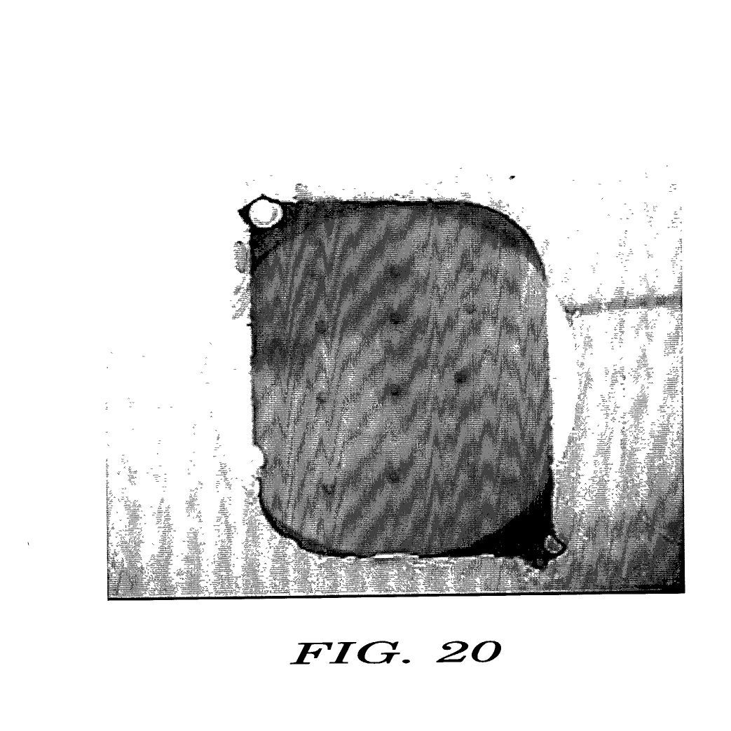

Fluidic dye experiments were also performed to investigate bubble-induced acoustic mixing in a 4-up biochip chamber. As shown in FIGS. 1-2 and 17-20, a PZT disk was adhered to the top surface of a polypropylene layer, which was pre-drilled with a number of air pockets (0.5 mm diameter and 0.5 mm depth) on the opposite side. These pockets were inside a reaction chamber, facing an array of oligonucleotide probes dispensed on a pre-treated glass slide. A double-sided adhesive tape (3M, 9490LE) was used to bond the polypropylene layer with the glass slide and served as a spacing gasket to define the shape and dimension of the chamber. The PZT was driven at 5.3 kHz and peak-to-peak 5 V. This experiment (FIGS. 17-20) shows that rapid mixing can be achieved across the whole chamber within 1.75 min, while the mixing based on pure diffusion (i.e., without acoustic mixing) takes about 1 hr for the same chamber.

### Example 4

The efficacy of acoustic mixing was also tested in an actual hybridization protocol. For this hybridization test, a 2-oligo array chip in a 4-up format was used. The array density for each chamber area was approximately 234 testing oligos and over 80 positive controls. The 2 oligo slide is made up of two oligos (NEO and YJEK) and a positive control. Both NEO and YJEK are bacterial oligonucleotides that were Cy3 labeled. The sequence of the NEO probe is GCGTTGGCTACCCGTGATATTGCTGAAGAG with 5′ amine. The sequence of the YJEK probe is TTTGTAGATTAGCACTGGAACTGGCACCGC with 5′ amine. These oligo probes were arranged in a uniform pattern across the entire slide. The dispense design of having one large 2 oligo dispense and the continuous repeating of the 2 oligos allows for comparisons across the entire array area, which enables seeing edge effects as well as hot and cold spots. This is critical in understanding the homogeneity of the entire array area.

A fluorescently labeled oligonucleotide target solution, which has 50% formamide, 6×SSPE, and Cy3 labeled targets was prepared. The targets were the complements to the NEO and YJEK oligo-probes on the slide. This focuses on the hybridization step of the assay and removes any variation caused by the detection system, such as TSA or Strep avidin. By using direct-labeled target, the 2 oligo slides are ideal in focusing strictly on how the mixing affects the uniformity of the signal across the entire array. This platform can then be used to compare different mixing platforms to non-mixing platforms to better understand how mixing improves the uniformity of the oligo signal as well as which mixing platform works best.

Hybridization of a 10 nM solution of the target in 6×SSPE was carried out. Acoustic mixing was applied to one of the 4up chambers, while static hybridization was performed in another chamber in the same chip. During hybridization, the chip was held at 37° C. Hybridization was carried out for 2 hr while driving the PZT crystal at 5.3 kHz at an average power of 2 mW. Following the hybridization, the polypropylene layer was peeled off from the glass slide, which was subsequently washed with TRIS/Sodium Chloride/Tween solution (TNT) for 30 minutes at 42° C. and then 3×water. The glass slide was scanned using an Axon scanner.

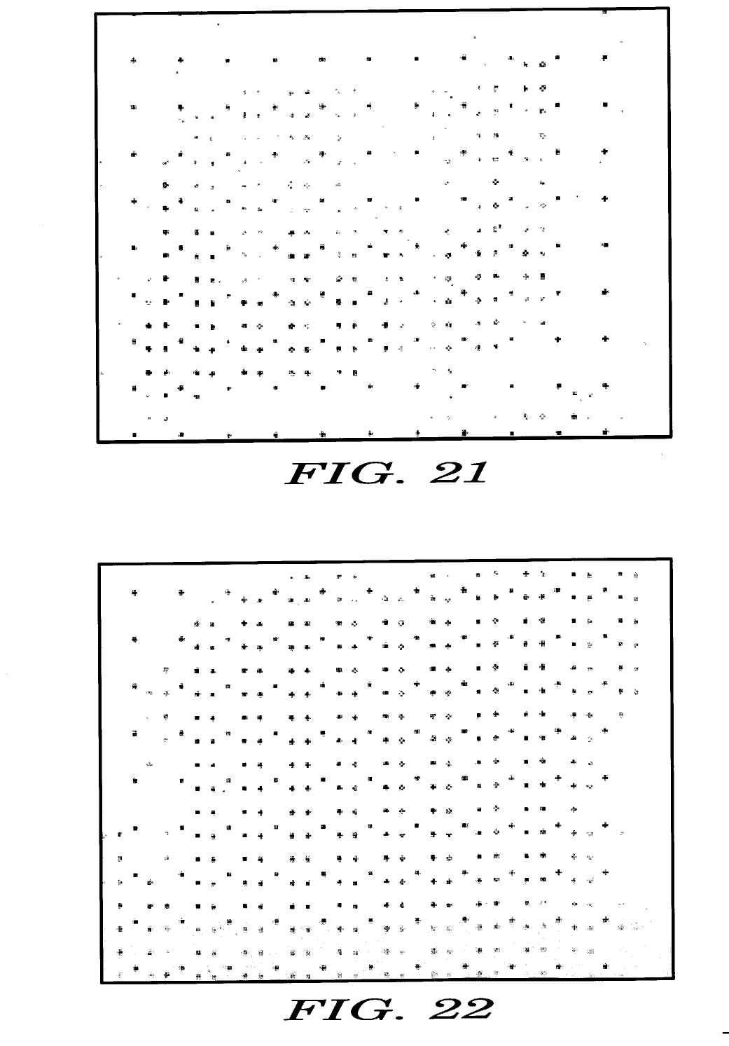

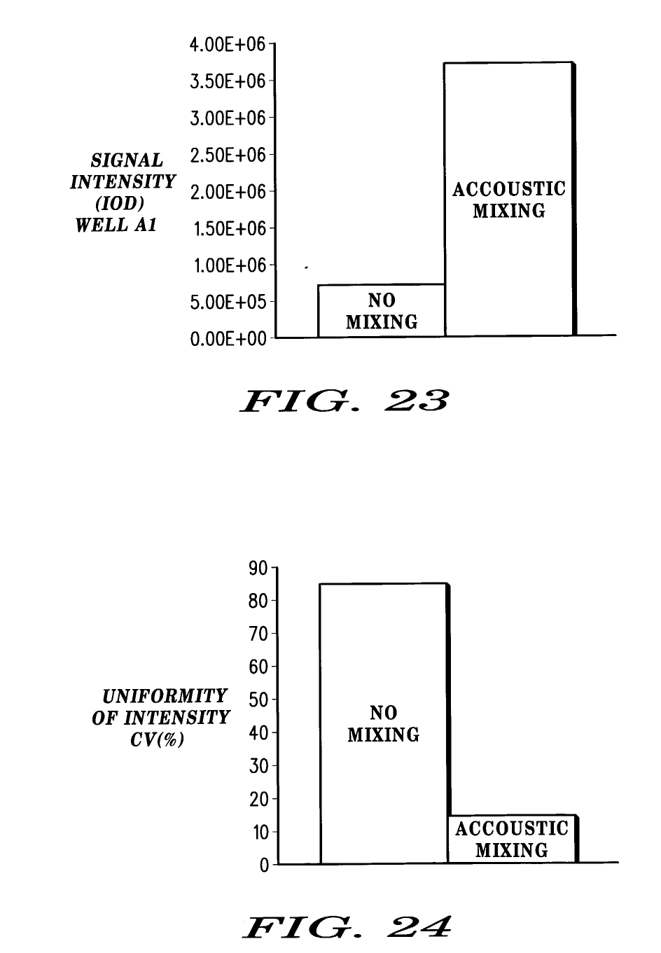

The resulting fluorescent scanning images are shown in FIGS. 21 and 22. Fluorescent intensity data for the mixing array and the non-mixing array (static hybridization reaction) were analyzed. As shown in FIGS. 23 and 24, the average intensity of the mixing array is five times more than that of the static hybridization array, and signal uniformity (co-variance) is greatly improved by implementing acoustic mixing. These results indicate that hybridization reactions in oligonucleotide array formats can generally be affected by the level of mixing of the target ligand, which was expected. Efficient and effective acoustic mixing can ensure maximal presentation of the sample targets to the array, and thus significantly improve hybridization efficiency and quality.

### Example 5

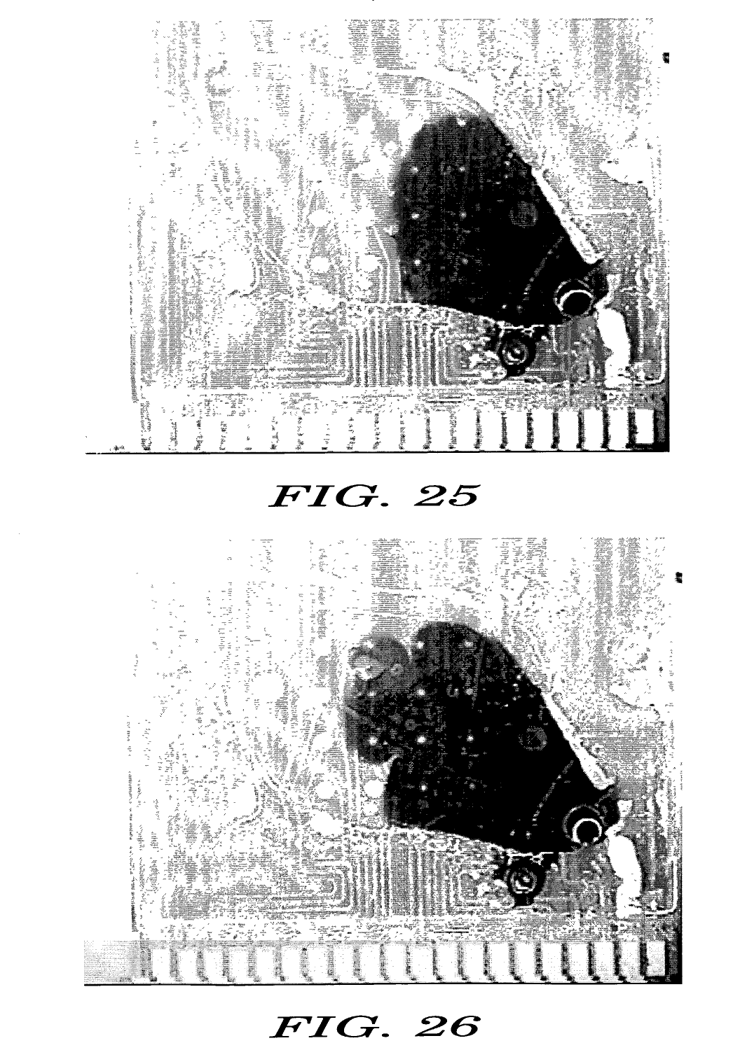

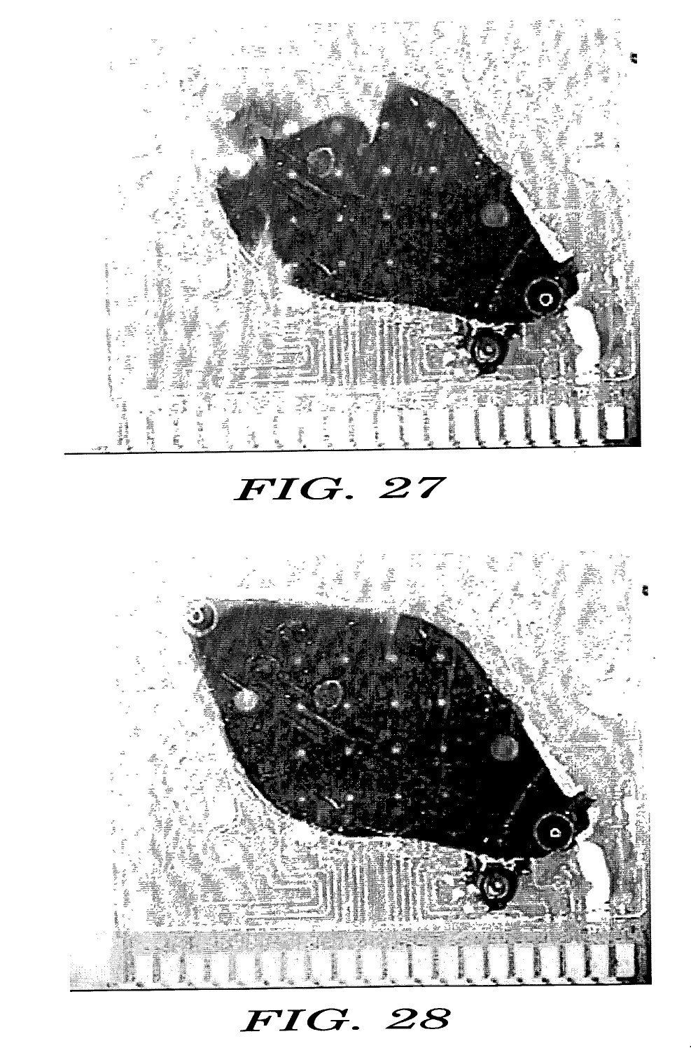

Bubble-induced acoustic mixing experiments were also performed in an e-Sensor™ (Clinical Micro Sensor Inc.) device that is a 16-pad PCB chip. A number of air pockets (0.5 mm diameter and 0.5 mm depth) were first drilled on the electrode side of the PCB. A PZT disk was glued on the backside of the PCB. A plastic layer with fluidic inlet and outlet holes was attached to the PCB with a double-sided adhesive tape to form a reaction chamber. The chamber was filled half space with DI water and the other half with a red dye solution (a mixture of phenolphthalein and sodium hydroxide solution). The frequencies employed were around 3.5 kHz (sinusoidal sound wave) with 5 V peak-to-peak amplitude. This experiment (FIGS. 25-28) showed that rapid mixing was achieved across the whole chamber within 2 min and 30 sec.

We have developed a bubble-induced acoustic mixing technique that is based on the principle of cavitation microstreaming. From fluidic experiments, we visualized that air bubbles resting on a solid surface and set into vibration by the sound field generated steady circulatory flows. By engineering bubbles and their distribution, we demonstrated that rapid and uniform dye mixing was achieved in a variety of devices, including plastic PCR mixing chamber, 4-up biochip chambers, and CMS e-Sensor devices. Preliminary hybridization tests in a 2-oligo array showed that the acoustic mixing significantly improves the signal intensity and uniformity within a short period of time (compared to the standard protocol). This mixing technique has many advantages over the mixing mechanisms of the prior art, including simple mixing apparatus, easy to implement (can be easily coupled to existing devices and systems), low power consumption (2 mW), and cost-effective. Moreover, this technique is particularly attractive for handheld electronic-driven DNA analysis instrument.

### Example 6

A microchannel with an air pocket micropump was integrated with an eSensor™ PCB (printed circuit board) substrate, schematically depicted in FIG. 3. The channel pattern was cut out of a double sided adhesive film 34, which was then placed between the PCB substrate 36 and a plastic cover plate 38. The channel pattern was aligned with the DNA capture probes so that the entire DNA array was sitting on the bottom of microchannel 30. The height of the channel is defined by the thickness of the adhesive film, which was approximately 200 μm, resulting in a channel volume of 20μL.

The channel was integrated with an air pocket micropump 26 that is similar to the bubble pump developed by Burns et al. WO 99/17093 To construct the air pocket micropump 26, a thin-film resistive heater 28 was deposited on the plastic cover plate 38 using a shadow mask (not shown). The shadow mask was made by deep reactive ion etching through a silicon wafer. Conventional photoresist processes (metal wet etching, and lift-off processes) cannot be used with plastic, although other materials for the substrate and cover plate may be used such that these other processes may be used. The placement of the shadow mask over plastic substrate and subsequent exposure to a sputtered metal source (e.g., Ti/Au) resulted in the formation of resistive metal lines on the plastic substrate. The plastic substrate serves as cover layer for the channel on the PCB substrate.

Two e-Sensor™ (manufactured by Clinical Micro Systems Inc., Pasadena Calif.) channel devices were used in this example, one with and one without an integrated air pocket pump. In order to avoid the problem of the expanded air heating up the liquid, the air pocket was placed at a distance from the liquid sample channel. It will be appreciated that other integrated heaters (such as microwave) and other pumping systems may be used without exceeding the scope of the present invention. For example, and without limitation, thick paste and green-sheets may be used to construct a microfluidic device with an integrated resistive heater, as described above. Having the heating elements above the air pocket prevents cross-talk, and reduces the amount of power consumption.

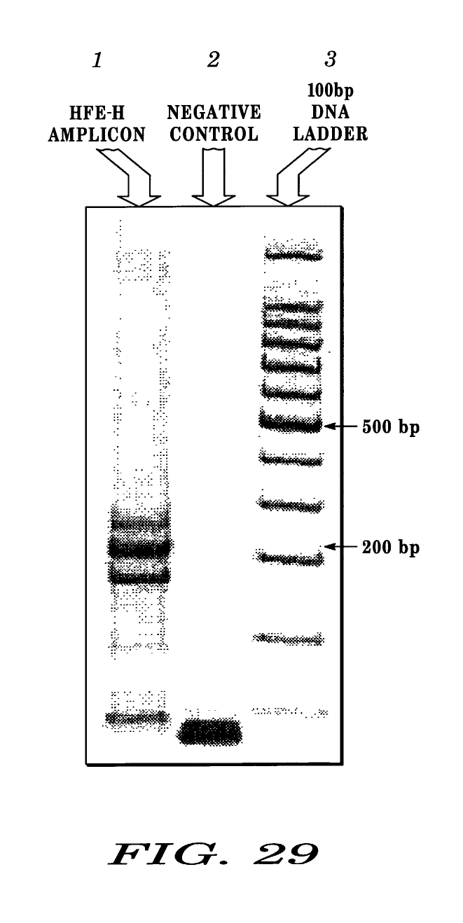

A HFE-H assay, as developed by Motorola Life Sciences, was chosen as the model assay. The DNA target solution containing the HFE-H polymorphism was amplified from human genomic DNA characterized for HFE genotype. The HFE samples were genotyped by asymmetrically amplifying 100 ng of human genomic DNA by PCR (3-primer PCR) to obtain mainly single-stranded amplification product. Cycling parameters were: 95° C. (3 min) to denture human DNA, followed by 40 cycles (94° C. for 45 sec, 58° C. for 55 sec, and 72° C. for 6 sec), and ending with 72° C. for 6 min to extend all unfinished DNA ends. The DNA amplicon (200bp) was confirmed by gel electrophoretic analysis as shown in FIG. 29.

After PCR and mixing with the hybridization solution (ratio 1:3), the amplicon solution was then pipetted into the inlet reservoir and flowed through the channel. The inlet reservoir was then sealed using an adhesive tape while the outlet reservoir was left open for air venting. This allows the pressure generated by the air pump to act only in the direction to the outlet port. Note that the outlet reservoir can be designed to be within a large containing chamber and a small outlet port so minimal evaporation of the sample solution is occurs (not shown here). Subsequently, the chips were plugged into an electronic control board. The control system continuously scanned the electrodes/DNA probes in the channel during the hybridization that occurs at room temperature (see U.S. Ser. No. 09/993,342, expressly incorporated herein by reference). For comparison purposes, we also performed hybridization reaction in a conventional diffusion-based chamber (70 μL) and two PZT-acoustic mixing chambers (where acoustic energy was used to generate microstreaming and hence enhance mixing in the chamber, as described above).

During the hybridization, the resistive heater was turned on and off every 10 minutes. The electrical resistance of the thin film heater was 45 ohm. The voltage applied to the heater was 4V. When the heater was turned on, the air trapped inside the pocket was heated up and generated an increased pressure that in turn moved the fluid in the channel towards the outlet reservoir. The fluid element can move a distance of 10 mm, but this distance can be increased by increasing the volume of the air pocket. However, a smaller air pocket allows a fast initiation of the pumping motion, since higher values of pressure are achieved more quickly. The maximum temperature reached near the heater is approximately 100° C. measured by a thermal coupler. By turning the heater off, the air pressure in the pocket decreased driving the flow back to the inlet. This flow oscillation allows fluid elements flowing across every single electrode/DNA probe. As a result, the chance of the target DNA in the solution to bind with the complementary DNA probe is significantly increased

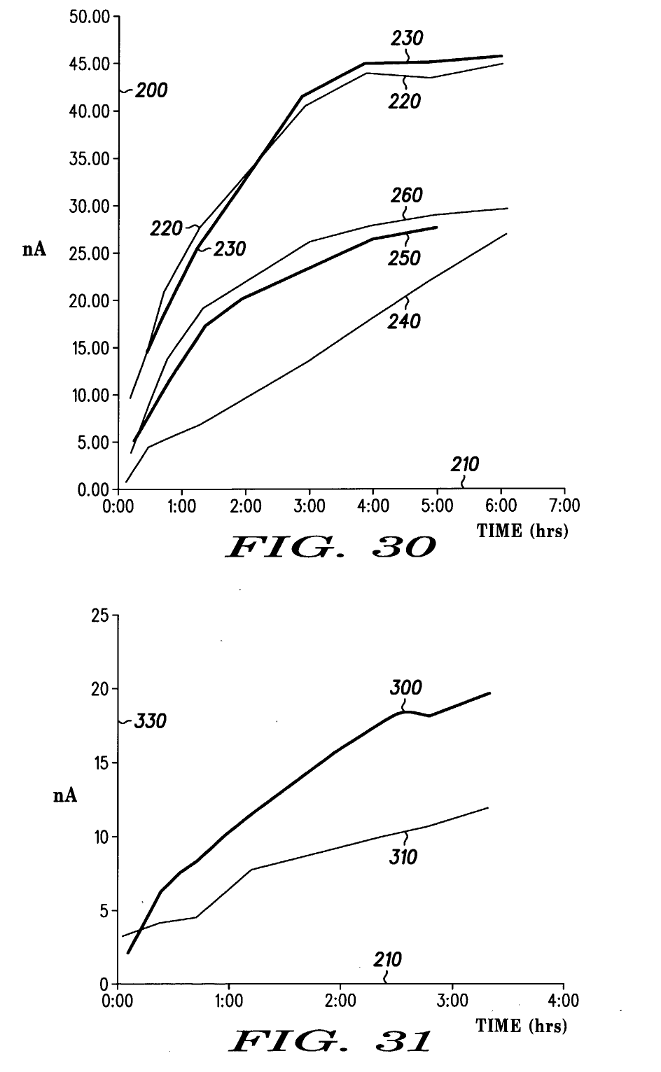

Kinetic data of target binding to sensor electrodes was collected by measuring electrochemical signal as a of function of time. FIG. 30 summarizes the hybridization kinetics results for the array channels with pumps 220, 230, conventional diffusion-based chamber 240, and PZT acoustic mixing chambers with 10V 250 and 20V 260. Each data point is the mean value collected from four electrodes with identical DNA capture probes. Note that y-axis 200 is the measurement of the faradaic current from the electrode in nanoamperes, while x-axis 210 is time in hours. The faradaic current is directly proportional to the number of ferrocene moieties (i.e., ETM) immobilized at the electrode surface that in turn is proportional to the number of target nucleic acid molecules. Faradaic current is the current specifically generated as a result of reduction or oxidation of the ferrocene ETM. Referring to FIG. 30, biochannels with oscillating micropumps signficantly improve hybridization kinetics as compared to the diffusion-based chamber and the acoustic mixing chamber. The hybridization in the channels (with pumps) reaches saturation within 3-4 hrs, while other methods took much longer. Moreover, despite its small volume of target solution, the saturation signal in the channels with micropumps is much higher than that of the acoustic mixing chips.

### Example 7

In a second experiment, we performed HFE-H assay in channels with and without integrated micropumps. In this second experiment we synthesized DNA oligonucleotides (70 bp) that served as target mimics, rather than the amplicon we used in the above experiments. Additionally, the pump was only turned on and off every 30 min here. As shown in FIG. 31, the channel with an integrated micropump 300 showed better hybridization kinetics over the channel without micropump 310.Note that y-axis 330 is the measurement of the faradaic current from the electrode in nanoamperes, while x-axis 210 is time in hours.

A DNA array channel with an integrated micropump not only allows reduced sample consumption, but also accelerates the hybridization kinetics by providing convectional oscillation flow along the channel. Oscillation flow along the channel results in: (1) enhanced mixing between the target DNA and DNA capture probes on the surface, which in turn will accelerate the hybridization process. Without wishing to be bound by any particular theory, although pressure-driven oscillation flow cannot change the fact that there is still a thin diffusion layer above the capture probe, the oscillation flow can increase the

DNA concentration gradient in the z-direction, and therefore, decrease depletion effects. This pumping-induced mixing enhancement results in faster hybridization in the biochannel with an integrated pump than without a pump (FIG. 31); (2) a locally focused hybridization reaction, since every target DNA is forced to pass by each capture probe along the narrow channel, and as a result each capture probe “sees” more of the target DNA within the diffusion width in the y-direction. It is believed, again without wishing to be bound by any particular theory, that this focusing effect is responsible for the high initial current signal in the biochannels compared to the diffusion-based chamber and acoustic mixing chamber (FIG. 30).

We have successfully demonstrated an electrochemical detection based DNA array channel with integrated micropump results in enhanced hybridization kinetics. This new platform is not only suitable for overcoming the inferior performance of conventional diffusion-based hybridization (large sample volume consumption and lengthy hybridization process), but is also easy to integrate with the front-end sample preparation in microfluidic components. Due to the mixing enhancement in the zdirection and target focusing effect in y-direction, the array channel with integrated micropump has demonstrated significant hybridization kinetics acceleration over other systems.

The hybridization enhancement technique of the present invention using oscillation flow in a DNA array channel has many advantage over other systems, including significant increases of hybridization kinetics, totally integrated and self-containing design, easy implementation (can be easily coupled to existing DNA chips), low power consumption (˜100 mW), and cost effectiveness. Moreover, this technique is particularly attractive for handheld electronic driven DNA analysis instrument.

What is claimed is:

1. A method of mixing a sample in a microfluidic cavity comprising:
(a) introducing said sample into a device comprising a microfluidic cavity comprising:
(i) at least one gas pocket; and
(ii) a substrate comprising at least one biological binding molecule; and
(b) altering the volume of said gas pocket to mix said sample such that said target analyte binds to said biological binding molecule.

2. The method according to claim 1, wherein said gas pocket further comprises a heater and said altering is done by heating and cooling said gas pocket.

3. The method according to claim 1, wherein said altering is done by the application of sonic waves to said microfluidic cavity.

4. The method according to claim 1, wherein said device further comprises a PZT film and said altering is done by oscillating said gas pocket with sonic waves.

5. The method according to claim 1, wherein said microfluidic cavity is a channel in a serpentine configuration, and said channel comprises a plurality of biological binding molecules distributed therein.

6. The method according to claim 1, wherein said microfluidic cavity is a microfluidic chamber, and said microfluidic chamber comprises a plurality of biological binding molecules distributed therein.

7. The method according to claim 1, wherein said microfluidic cavity comprises an array of different biological binding molecules.

8. The method according to claim 1, wherein said substrate comprises electrodes with biological binding molecules attached thereto.

9. The method according to any one of claims 1 or 5-8, wherein said biological binding molecules are nucleic acids.

10. The method according to claim 1, wherein said substrate comprises a material selected from the group consisting of ceramics, printed circuit board, and glass.

11. The method according to claim 1, wherein said microfluidic cavity has an indentation within a wall thereof, such that upon introduction of said sample, said gas pocket is at least partially defined between said indentation and said sample.

12. The method according to claim 1, wherein said microfluidic cavity has a plurality of indentations within one or more walls thereof, such that upon introduction of said sample, a plurality of gas pockets are at least partially defined between said indentations and said sample.

13. A method of detecting a target analyte in a sample comprising:
(a) introducing said sample into a device comprising a microfluidic cavity comprising:
(i) at least one gas pocket; and
(ii) a substrate comprising at least one biological binding molecule;
(b) altering the volume of said gas pocket to mix said sample such that said target analyte binds to said biological binding molecule; and
(c) detecting the presence of said target analyte.

14. The method according to claim 13 wherein said gas pocket further comprises a heater and said altering is done by heating and cooling said gas pocket.

15. The method according to claim 13 wherein said altering is done by the application of sonic waves to said microfluidic cavity.

16. The method according to claim 13, wherein said device further comprises a PZT film and said altering is done by oscillating said gas pocket with sonic waves.

17. The method according to claim 13, wherein said microfluidic cavity is a channel in a serpentine configuration, and said channel comprises a plurality of biological binding molecules distributed therein.

18. The method according to claim 13, wherein said microfluidic cavity is a microfluidic chamber, and said microfluidic chamber comprises a plurality of biological binding molecules distributed therein.

19. The method according to claim 13, wherein said microfluidic cavity comprises an array of different biological binding molecules.

20. The method according to claim 13, wherein said substrate comprises electrodes with biological binding molecules attached thereto.

21. The method according to any one of claims 13-20, wherein said biological binding molecules are nucleic acids.

22. The method according to claim 13-20, wherein said detecting is selected from the group consisting of detecting fluorescence, detecting the change in an electrical property, and detecting the an electron transfer moiety.

23. The method according to claim 13, wherein said substrate comprises a material selected from the group consisting of ceramics, printed circuit board, and glass.

24. The method according to claim 13, wherein said microfluidic cavity has an indentation within a wall thereof, such that upon introduction of said sample, said gas pocket is at least partially defined between said indentation and said sample.

25. The method according to claim 13, wherein said microfluidic cavity has a plurality of indentations within one or more walls thereof, such that upon introduction of said sample, a plurality of gas pockets are at least partially defined between said indentations and said sample.

26. A method for mixing a solution within a microfluidic chamber comprising:
(a) introducing a liquid into a microfluidic cavity, such that a gas pocket exists within the liquid; and
(b) applying sonic waves to said gas pocket, thereby resulting in the mixing of said solution within said microfluidic cavity.

27. The method according to claim 26, wherein said microfluidic cavity has an indentation within a wall thereof, such that upon introduction of said liquid said gas pocket is at least partially defined between said indentation and said liquid.

28. The method according to claim 26, wherein said gas pocket is a gas bubble within said microfluidic cavity.

29. The method according to claim 26, wherein said microfluidic cavity is a microfluidic chamber.

30. The method according to claim 29, wherein said microfluidic chamber further comprises an array of probe molecules enclosed therein.

31. The method according to claim 30, wherein said probe molecules are in contact with an electrode, such that an interaction with said probe molecule by a target molecule may be detected by a change in an electrical property.

32. A method of detecting the presence of a target molecule in a sample solution:
(a) introducing a sample solution into a microfluidic chamber such that a gas pocket exists within the solution, wherein said microfluidic chamber comprises an array of probe molecules;
(b) applying sonic waves to said gas pocket, thereby resulting in the mixing of said solution within said microfluidic chamber; and
(c) detecting an interaction of a target molecule form said sample solution with a probe molecule of said array, thereby detecting the presence of said target molecule in said sample solution.

33. The method according to claim 32, wherein said target molecule is labeled with a detectable reporter.

34. The method according to claim 33, wherein said detectable reporter is selected from the group consisting of radioactive, fluorescent, and electrochemical.

35. A method of detecting a target analyte in a sample comprising:
(a) introducing said sample into a device comprising a microfluidic cavity, wherein said microfluidic cavity is a channel in a serpentine configuration, and said channel comprises a plurality of biological binding molecules distributed therein;
(b) oscillating the sample within said channel, such that said target analyte binds to said biological binding molecule; and
(c) detecting the presence of said target analyte.

36. The method according to claim 35, wherein said detecting is selected from the group consisting of detecting fluorescence, detecting the change in an electrical property, and detecting the an electron transfer moiety.

37. A method of mixing a sample in a microfluidic cavity comprising:
(a) introducing said sample into a device comprising a microfluidic cavity, wherein said microfluidic cavity is a channel in a serpentine configuration; and
(b) oscillating the sample within said channel.

38. A microfluidic device comprising:
(a) a body defining a microfluidic chamber having an indentation within a wall thereof;
(b) a means for applying sonic frequency to said microfluidic cavity.

39. The microfluidic device according to claim 38, wherein said means for applying sonic frequency is a PZT film.

40. The microfluidic device according to any one of claims 38 or 39, wherein said microfluidic cavity has a plurality of indentations within one or more walls thereof.

41. A microfluidic device comprising:
at least one microfluidic cavity having an indentation therein, wherein said microfluidic cavity has an indentation within a wall thereof, such that upon introduction of a liquid a gas pocket is at least partially defined between said indentation and said liquid, whereby application of sonic waves to the gas pocket results in mixing of the liquid within said at least one microfluidic cavity.

42. The microfluidic device according to claim 41, wherein said microfluidic cavity is a microfluidic chamber.

43. The microfluidic device according to claim 42, wherein said microfluidic chamber further comprises an array of probes molecules enclosed therein.

44. The microfluidic device according to claim 43, wherein said probe molecules are in contact with an electrode, such that an interaction with said probe molecule by a target molecule may be detected by a change in an electrical property.

45. The microfluidic device according to claim 44, wherein said electrical property is impedance.

46. The microfluidic device according to claim 41 further comprising:
means for applying sonic waves to said microfluidic cavity.
